(19) United States
(12) Patent Application Publication
(10) Pub. No.: US 2025/0236876 A1
(43) Pub. Date: Jul. 24, 2025

(54) ENGINEERED YEAST FOR EFFICIENT AND RAPID SYNTHESIS OF ERYTHRITOL AND CONSTRUCTION METHOD THEREOF

(71) Applicant: Shanghai Jiao Tong University, Shanghai (CN)

(72) Inventors: Hairong Cheng, Shanghai (CN); Shuo Xu, Shanghai (CN)

(21) Appl. No.: 18/772,022

(22) Filed: Jul. 12, 2024

(30) Foreign Application Priority Data
Jan. 22, 2024 (CN) 202410087435.8

(51) Int. Cl. C12N 15/81; C07K 14/475; C07K 14/705; C12N 1/16; C12N 9/12; C12N 9/88; C12N 9/90; C12P 7/18

(52) U.S. Cl. CPC C12N 15/815; C07K 14/475; C07K 14/705; C12N 1/16; C12N 9/1205; C12N 9/88; C12N 9/90; C12P 7/18; C12Y 207/01002; C12Y 207/01011; C12Y 401/02013; C12Y 501/03001; C12Y 503/01006

(57) ABSTRACT

Disclosed are an engineered yeast for efficient and rapid synthesis of erythritol and a construction method thereof. Yarrowia lipolytica is used as a synthetic chassis for genetic improvement. A method for synthesizing erythritol is as follows: using glucose as a carbon source, and a nitrogen source and an inorganic salt as raw materials, sterilizing a medium, cooling the sterilized medium before inoculating yeast Yarrowia lipolytica, performing continuous fermentation or fed-batch fermentation under the condition of oxygen supply, and purifying erythritol from a fermentation broth. Under the condition of continuous feeding, the yield of erythritol is more than 350 g/L, and the production efficiency is more than 4.5 g/L·h, nearly 100% higher than that of a comparative strain.

### CROSS-REFERENCE TO RELATED APPLICATIONS

This application claims priority of Chinese Patent Application No. 202410087435.8, filed on Jan. 22, 2024, the entire contents of which are incorporated herein by reference.

### SEQUENCE LISTING

The instant application contains a Sequence Listing with 50 sequences, which has been submitted electronically in XML format and is hereby incorporated herein by reference in its entirety. Said XML copy, created on Jul. 12, 2024 is named XZLWZJ-US-1-04-KAG50295.xml, and is 73 kbytes in size.

### TECHNICAL FIELD

The present disclosure belongs to the technical field of food biotechnologies, and relates to an engineered yeast for efficient and rapid synthesis of erythritol and a construction method thereof, and more specifically, relates to a construction method for efficient synthesis of erythritol by fermentation by means of metabolic engineering using Yarrowia lipolytica as a chassis microorganism, the method being utilized to obtain a proprietary Yarrowia lipolytica strain capable of synthesizing erythritol by fermentation of glucose as a carbon source, and the strain being fermented to synthesize erythritol.

### BACKGROUND

Excessive consumption of high-calorie table sugar and glucose fructose syrup may be prone to health problems of hyperglycemia, obesity and hyperlipidemia, and therefore, it is particularly important to prevent these health problems in diet. In recent years, sugar-reduced and sugar-controlled foods are attractive to consumers. Sugar-reduced products typically contain low-calorie polyols such as xylitol, mannitol, maltitol and erythritol. Among polyol products, erythritol, as a natural sweetening food additive, has been widely applied in food industry due to its unique characteristics, such as higher acceptable daily intake and safety, lower hygroscopicity, and no aftertaste than any other sweetening agents of sugar alcohols. As synthesizing erythritol chemically is low in yield, high in complexity and harsh in operating conditions, the chemical synthesis of erythritol offers no advantages, while synthesizing erythritol by fermentation is low in cost, eco-friendly and safe, which has become the only way for commercialized production of erythritol. Erythritol is mainly obtained by glucose fermentation of hyperosmotic yeast, and the optimization for production parameters of erythritol is mainly centralized on strain screening, mutation breeding, medium optimization and culture mode. Currently, the maximum yield, conversion rate and production efficiency are approximately 240 g/L, 0.60 g/g and 2.84 g/L·h, respectively (Jeya, M., Lee, K. M., Tiwari, M. K., Kim, J. S., Gunasekaran, P., Kim, S. Y, Kim, I. W., Lee, J. K., 2009. Isolation of a novel high erythritol-producing Pseudozyma tsukubaensis and scale-up of erythritol fermentation to industrial level. Appl. Microbiol. Biotechnol. 83, 225-31). The strain used is Pseudozyma tsukubaens, and despite having high yield and production efficiency, the strain does not belong to the strains generally recognized as safe (GRAS) approved by the food and drug administration (FDA) of the United States or by China, which limits its use.

Yarrowia lipolytica has received more attention due to its excellent genetic manipulability, extensive substrate utilization and recognized safety (Bilal, M., Xu, S., Iqbal, H. M. N., Cheng, H., 2020. Yarrowia lipolytica as an emerging biotechnological chassis for functional sugars biosynthesis. Critical Reviews in Food Science and Nutrition. 61, 535-552.). After mutagenesis and optimization of culture conditions, the yeast has a maximum production, conversion rate (yield) and productivity of erythritol of 231.2 g/L (Li, S., Zhang, Y, Li, L., Yuan, Y, Sun, H., Xing, X.-H., Wang, X., Zhang, C., 2023. Establishment of picodroplet-based co-culture system to improve erythritol production in Yarrowia lipolytica. Biochem. Eng. J. 198.), 0.67 g/g (Mirończuk, A. M., Dobrowolski, A., Rakicka, M., Rywinska, A., Rymowicz, W., 2015. Newly isolated mutant of Yarrowia lipolytica MK1 as a proper host for efficient erythritol biosynthesis from glycerol. Process Biochem. 50, 61-68.) and 2.51 g/L·h (Wang, N., Chi, P., Zou, Y, Xu, Y, Xu, S., Bilal, M., Fickers, P., Cheng, H., 2020. Metabolic engineering of Yarrowia lipolytica for thermoresistance and enhanced erythritol productivity. Biotechnol Biofuels. 13, 176), respectively. Although the production and yield of erythritol from existing Yarrowia lipolytica strains have reached industrial levels, the production efficiency (productivity) is still low, with a longer fermentation time and higher energy consumption, which means that the production costs increase due to the longer fermentation time. Accordingly, it is necessarily to improve the yield by means of metabolic engineering.

The raw materials reported for the production of erythritol with Yarrowia lipolytica as a strain include glucose or glycerol. In most of the literature, glycerol is taken as a raw material, because it is believed that glycerol can be derived from waste materials by biodiesel processing and is of low value. Therefore, it is believed that the use of glycerol, a waste material by biodiesel processing, as a carbon source for fermentation to produce erythritol has the advantage in raw material. On the basis of this, Mirończuk et al. applied glycerol for the first time as a carbon source for metabolic engineering of erythritol to improve Yarrowia lipolytica, and to enhance the production of erythritol by utilizing glycerol to transport the cell membrane acceleratedly. The productivities of erythritol overexpressed by glycerol kinase (GK or GUT1) and co-expressed by GK with glycerol-3-phosphate dehydrogenase (GDH or GUT2) are 24% and 35% higher than those of the comparative strain, respectively, and 150 g/L of glycerol can be consumed completely in 44-48 h, a significant reduction in time over the comparative strain (Mironczuk, A. M., Rzechonek, D. A., Biegalska, A., Rakicka, M., Dobrowolski, A., 2016. A novel strain of Yarrowia lipolytica as a platform for value-added product synthesis from glycerol. Biotechnology for Biofuels. 9:1-12). Crude glycerol is acceptable as a cheap carbon source for the production of chemical products, but the current erythritol is mainly used in food industry, which has higher requirements for raw materials. The crude glycerol produced as waste materials from biodiesel processing contains a variety of impurities, which does not meet the food-grade requirements, and in fact, this crude glycerol is not approved as a raw material in the production of food-grade erythritol. At present, the main raw material for industrial production of erythritol is still food-grade glucose powder or liquid glucose, and therefore, it is particularly important to develop a yeast chassis for efficient production of erythritol by utilizing glucose, which can shorten the fermentation time and improve the production efficiency.

In a patent filed by the inventors in 2020 (patent application number: 2020100692506.6, patent title: construction method for recombinant Yarrowia lipolytica for synthesizing erythritol and strains thereof, inventors: Cheng Hairong, Wang Nan, and Chi Ping), partial gene expressions involved in the erythritol synthesis pathway of Yarrowia lipolytica are improved by metabolic engineering and some of the genes related to the synthesis of by-products and some of the genes related to the utilization of the products are knocked out, enhancing the efficiency of “synthesis machine”, and improving the yield and efficiency of synthesizing erythritol. The patented strain CGMCC No. 19351 obtained is fermented in a 150 L fermentation tank to synthesize erythritol with a maximum yield of 198.4 g/L, a conversion rate of 62%, a fermentation time of 85 h, and a production efficiency of 2.33 g/L·h, and without synthesizing the by-product mannitol and without utilizing the product erythritol. Although the technical parameters have been improved over the previous technology, there is still a lot of study for improvement and optimization, for example, the conversion rate (yield) and production efficiency (productivity or production intensity) still needs to be greatly improved, and the fermentation time is still long, which needs to be greatly reduced to save costs and save energy. It is very important to overcome these disadvantages, so that a great deal of work is carried out in the present disclosure.

### SUMMARY

Aiming at the shortcomings of an existing strain for synthesis of erythritol by glucose fermentation, the present disclosure provides an engineered yeast for efficient and rapid synthesis of erythritol and a construction method thereof, and the construction method is employed to construct an engineered strain of Yarrowia lipolytica for efficient and rapid synthesis of erythritol by fermentation, and the strain is used to ferment carbon sources such as glucose to synthesize erythritol and to isolate and purify erythritol from an erythritol-rich fermentation broth.

In the present disclosure, it is found that the rapid and efficient transportation of substrates into cells is an important research point for the development of efficient microbial cell factories, and that only if the substrate raw materials enter the cells efficiently and rapidly, can an intracellular “synthesis machine” obtain sufficient raw materials to synthesize products. At the same time, it is also very important to strengthen the collaboration of the intracellular “synthesis machine”. When the substrate raw materials are efficiently transported into the cells, if the synthesis machine fails to synthesize products in a timely and rapid manner, it will lead to the accumulation of substrate, resulting in feedback inhibition on the transportation of the substrate. Also, the products after synthesis need to be transported out of the cells in a timely manner, otherwise feedback inhibition is possible. Therefore, it is necessary to tightly integrate the transporter proteins engineering (including the transporter proteins for the transfer of raw materials as well as the transporter proteins for the output of products) with the pathway engineering in order to realize the efficient synthesis of the product erythritol, both of which are indispensable. In the present disclosure, this global design strategy rather than a previous approach in which only focusing on the intracellular erythritol synthesis pathway but not transporting pathway is employed to achieve efficient and rapid synthesis of erythritol.

In the present disclosure, an original patented Yarrowia lipolytica strain CGMCC No. 19351 synthesizing erythritol is improved with multiple strategies by means of synthetic biology. Strain CGMCC No. 19351, also known as Yarrowia lipolytica ery949-4Δ in Chinese invention patent with application number of CN2020100692506.6, was deposited to China General Microbiological Culture Collection Center (CGMCC) on Jan. 14, 2020, at the Institute of Microbiology, Chinese Academy of Sciences (IMCAS), No. 1, Beichen West Road, Chaoyang District, Beijing, with a deposition number of CGMCC No. 19351, and a genotype of: ery::HK::TKL1::TAL::EryPaseΔPGIΔArDHΔMDHΔEYD[overexpressed fructosekinase genes (HK), transketolase genes (TKL), transaldolase genes (TAL), and erythrose 4-phosphate phosphorylase genes (EryPase), as well as the knockdown of four genes: phosphorylated glucose isomerase (PGI), arabitol dehydrogenase (ArDH), mannitol dehydrogenase (MDH) and erythritol dehydrogenase genes (EYD)], and the background information and a construction method for the chassis strain are detailed in the China national invention patent with application number of CN2020100692506.6. In the present disclosure, the chassis CGMCC No. 19351 is taken as an original strain for improvement, and a new yeast of Yarrowia lipolytica CGMCC No. 28807 is obtained, which is capable of utilizing glucose as a carbon source to synthesize erythritol efficiently and rapidly.

In the present disclosure, by means of metabolic engineering improvement, further synergistic genetic manipulation is performed on the basis of chassis yeast CGMCC No. 19351, which, on the one hand, further enhances the efficiency of transporting the substrate carbon source such as glucose into chassis cells, on the other hand, further enhances the ability of intracellular erythritol synthesis, and on the other hand, enhances the efficiency of transporting product erythritol from intracellular to medium, thus enabling the new Yarrowia lipolytica strain obtained to synthesize erythritol efficiently and rapidly by fermenting glucose carbon source on the basis of the collaboration of these three aspects. Due to the retention of the properties of the original strain CGMCC No. 19351, such as no synthesis of by-products of mannitol and arabinitol and no utilization of erythritol, the new strain Yarrowia lipolytica CGMCC No. 28807 has better implementation effect than the original strain CGMCC No. 19351, and the achieved implementation effect is detailed in examples.

An objective of the present disclosure can be realized by the following technical solutions.

In a first aspect, the present disclosure provides an engineered yeast for efficient and rapid synthesis of erythritol, which is obtained by introducing genes related to the synthesis of erythritol by using a Yarrowia lipolytica strain as a chassis microorganism.

In an embodiment of the present disclosure, the Yarrowia lipolytica strain has a genome containing a deoxyribonucleic acid (DNA) sequence having 97% or more homology or similarity to a sequence of SEQ ID No. 1 and is capable of synthesizing erythritol.

Further, the Yarrowia lipolytica strain includes any one of Yarrowia lipolytica strains such as CGMCC 7326, Yarrowia lipolytica ery929 CGMCC No. 18478, and Yarrowia lipolytica ery929 CGMCC No. 19351. The Yarrowia lipolytica CGMCC 7326 strain is referred to the paper published by the inventors (Huiling Cheng et al. Identification, characterization of two NADPH-dependent erythrose reductases in the yeast Yarrowia lipolytica and improvement of erythritol productivity using metabolic engineering. Microbial Cell Factories, 2018, 17:133.).

Furthermore, the Yarrowia lipolytica strain is Yarrowia lipolytica ery929 CGMCC No. 19351.

The chassis strain Yarrowia lipolytica ery929 used in the present disclosure has been deposited to CGMCC on Jan. 14, 2020 at IMCAS, No. 1, Beichen West Road, Chaoyang District, Beijing, China, with the deposition number of CGMCC No. 19351.

In an embodiment of the present disclosure, the genes related to the synthesis of erythritol include one or more of the following genes:
(1) genes encoding glucose transporter proteins (GTPs), including GTPs1 (SEQ ID No. 2), GTPs2 (SEQ ID No. 3), GTPs3 (SEQ ID No. 4), GTPs4 (SEQ ID No. 5), etc;
(2) genes encoding erythritol synthases (ETs), including ETs1 (SEQ ID No. 6), ETs2 (SEQ ID No. 7), ETs3 (SEQ ID No. 8), ETs4 (SEQ ID No. 9), etc;
(3) genes encoding ribulose-5-P isomerase (RPI, SEQ ID No. 10);
(4) genes encoding ribulose-5-P epimerase (RPE, SEQ ID No. 11);
(5) genes encoding glucose kinase (GLK, SEQ ID No. 12);
(6) genes encoding erythritol transporter proteins (ETPs, SEQ ID No. 13);
(7) genes encoding fructose-6-P kinase (FPK, SEQ ID No. 14);
(8) genes encoding fructose-1,6-bisphosphate aldolase (FBA, SEQ ID No. 15);
(9) genes encoding growth factors (GFs, SEQ ID No. 16); and
(10) genes encoding growth factor-DNA-binding transcription factors (GFDBTFs, SEQ ID No. 17).

It is to be mentioned in particular that the ten gene types or 16 gene sequences (SEQ ID No. 2 to SEQ ID No. 17) described above are all derived from Yarrowia lipolytica itself, rather than any other yeasts or bacteria. An objective of introducing the above ten gene types or 16 gene sequences (SEQ ID No. 2 to SEQ ID No. 17) is to enable the chassis microorganism to acquire or enhance corresponding functions.

In an embodiment of the present disclosure, the genes for efficient synthesis of erythritol include: the genes (1), the genes (6), and at least one of the genes (2)-(5) and (7)-(10).

In some preferred examples, the yeast is obtained by introducing a gene expression cassette for which possibly related to efficient synthesis of erythritol as shown in the sequences of SEQ ID No. 2 to SEQ ID No. 17 by using a Yarrowia lipolytica strain as a chassis microorganism.

In a second aspect, the present disclosure provides an engineered yeast for efficient and rapid synthesis of erythritol, the yeast being a Yarrowia lipolytica strain with a deposition number of CGMCC No. 28807, and deposited to CGMCC on Nov. 14, 2023 at IMCAS, No. 1, Beichen West Road, Chaoyang District, Beijing.

In a third aspect, the present disclosure provides a construction method for an engineered yeast for efficient and rapid synthesis of erythritol, including the following steps:
A1. designing a gene expression cassette for efficient synthesis of erythritol and synthesizing the gene expression cassette,
A2. transforming the gene expression cassette synthesized in step A1 into Yarrowia lipolytica, and
A3. screening for Yarrowia lipolytica containing the gene expression cassette; or,
B1. combining a gene for efficient synthesis of erythritol as shown in any one or more of SEQ ID No. 2 to SEQ ID No. 17 with a promoter and a terminator of the gene to form a gene open reading frame (ORF), and
B2. screening for Yarrowia lipolytica containing the gene ORE.

In an embodiment of the present disclosure, in step A1, the gene expression cassette for the efficient synthesis of erythritol includes an upstream homologous integration arm sequence, a first promoter sequence, a gene sequence for efficient synthesis of erythritol as shown in any one or more of SEQ ID No. 2 to SEQ ID No. 17, a first terminator sequence, a second promoter sequence, a screening marker sequence, a second terminator sequence, and a downstream homologous integration arm sequence.

Further, in some examples, the gene expression cassette for the efficient synthesis of erythritol includes an upstream homologous integration arm sequence, a downstream homologous integration arm sequence, a promoter sequence, a terminator sequence, a screening marker sequence, and a gene sequence for the efficient synthesis of erythritol as shown in any one or more of SEQ ID No. 2 to SEQ ID No. 17.

In an embodiment of the present disclosure, the upstream and downstream homologous integration arm sequences are segments of DNA sequence from a Yarrowia lipolytica genome, and can insert a DNA sequence between the upstream and downstream homologous arms into homologous DNA sequences in the genome by homologous double crossover recombination. Commonly used homologous arms include an intA sequence, an intB sequence, an intC sequence, an intD sequence, an intE sequence, an intF sequence, etc., of Yarrowia lipolytica (Holkenbrink C, Dam M I, Kildegaard K R, et al. EasyCloneYALI: CRISPR/Cas9-based synthetic toolbox for engineering of the yeast Yarrowia lipolytica [J]. Biotechnology journal, 2018, 13(9): 1700543.), and these sequences are available in public databases such as National Center of Biotechnology Information (NCBI).

In an embodiment of the present disclosure, the promoter sequence is a segment of DNA sequence capable of initiating the transcription of a downstream gene, which can be an artificially synthesized promoter sequence such as the promoter sequences of UAS1B8, UAS1B16, hp4d, etc. (Blazeck et al. 2013. Generalizing a hybrid synthetic promoter approach in Yarrowia lipolytica. Appl Microbiol Biotechnol, 97:3037-3052.), or promoter sequences from genes of Yarrowia lipolytica such as a promoter sequence of TKL genes, a promoter sequence of TAL genes, a promoter sequence of P_{EXP}, etc.

In an embodiment of the present disclosure, the terminator sequence is a segment of DNA sequence capable of terminating the transcription of an upstream gene, such as a terminator sequence of cytochrome (CYC) genes (Tcyc1), a terminator sequence of alkaline protease (AEP) genes (Taep), and a terminator sequence of TKL genes, etc.

In an embodiment of the present disclosure, the screening marker sequence refers to antibiotic resistance genes such as hygromycin resistance genes, etc., or nutrient-selective genes such as lactase genes (Lac2, encoding a product to enable Yarrowia lipolytica to utilize lactose), ribitol dehydrogenase genes (RDH, encoding a product to enable Yarrowia lipolytica to utilize ribitol), uracil nucleotide synthetase genes 3 (URA3, encoding a product to enable ura3-deficient Yarrowia lipolytica to grow on a uracil-free basic medium), sucrase Suc2 genes, and mycophenolic acid screening marker (guaB), etc.

In an embodiment of the present disclosure, in step A1, the gene expression cassette contains a homologous integration arm, including upstream and downstream sequences.

In the present disclosure, an integral expression cassette (i.e., an integration expression vector) for genome integration is synthesized in vitro on the basis of the sequences of the genes to be expressed as described above (SEQ ID No. 2 to SEQ ID No. 17) in Yarrowia lipolytica. The integration expression vector contains a homologous integration arm sequence (including upstream and downstream segments), a promoter sequence, a terminator sequence, a screening marker sequence, and other necessary DNA elements. In the present disclosure, the homologous integration arm sequence is a segment of DNA sequence from a Yarrowia lipolytica genome, and can insert a DNA sequence between the upstream and downstream homologous arms into homologous DNA sequences in the genome by homologous double crossover recombination. The promoter sequence is a segment of DNA sequence capable of inducing the initiation of transcription of a downstream gene, which can be an artificially synthesized promoter sequence such as the promoter sequences of UAS1B8, UAS1B16, hp4d, etc. (Blazeck et al. 2013. Generalizing a hybrid synthetic promoter approach in Yarrowia lipolytica. Appl Microbiol Biotechnol, 97:3037-3052.), or promoter sequences from genes of Yarrowia lipolytica such as a promoter sequence of TKL genes, a promoter sequence of TAL genes, etc. The terminator is a segment of DNA sequence that can terminate the continued transcription of an upstream gene. The screening marker sequence refers to antibiotic resistance genes such as hygromycin resistance genes, etc., or nutrient-selective genes such as Lac2 (encoding a product to enable Yarrowia lipolytica to utilize lactose), RDH genes (encoding a product to enable Yarrowia lipolytica to utilize ribitol), URA3 (encoding a product to enable ura3-deficient Yarrowia lipolytica to grow on a uracil-free basic medium). A plasmid contains upstream and downstream homologous integration arm sequences, a promoter sequence, a target gene sequence (i.e., a gene sequence for efficient synthesis of erythritol), a terminator sequence, a screening marker sequence used in Yarrowia lipolytica, and other necessary DNA elements.

In an embodiment of the present disclosure, in step A2, a conversion method is referred to the paper published by Cheng Hairong, the present inventor: Journal of Functional Foods, 2017, 32:208-217.

In an embodiment of the present disclosure, in steps A3 and B2, the screening is performed in a medium containing screening markers such as mycophenolic acid. If the integration expression vector contains a lactase screening marker, after conversion, the yeast is placed on a lactose-containing yeast nitrogen base (YNB) basic medium for screening (6 g/L of YNB, 5 g/L of ammonium sulfate, 10 g/L of lactose, 15 g/L of agar powder, and a pH value of 6.0). If the integration expression vector contains a hygromycin resistance gene screening marker, after conversion, the yeast is placed on a hygromycin B-containing yeast extract peptone dextrose (YPD) medium for screening (10 g/L of glucose, 10 g/L of yeast extract powder, 5 g/L of peptone, 15 g/L of agar, 300 g/mL of hygromycin B, a pH value of 6.0). If the integration expression vector contains a mycophenolic acid screening marker, after conversion, the yeast is coated on a medium containing mycophenolic acid for screening (6.7 g/L of YNB, 5 g/L of ammonium sulfate, 20 g/L of glucose, 300 g/mL of mycophenolic acid, 15 g/L of agar powder, and a pH value of 6.5).

Further, the total RNA is extracted from a transformant (yeast cell containing overexpressed genes), and is subjected to reverse transcription for quantitative gene expression (qPCR); and if a gene expression increases significantly, it indicates that a target gene has been integrated into the Yarrowia lipolytica genome and expressed. A Cre/loxP system is adopted to recover the screening maker gene in the transformant (see references for the paper: J. Microbiol. Methods, 2003, 55, 727-737), and a specific recovery and screening method is described in detail in examples. A first target gene is integrated into the genome, and an engineered strain obtained after the screening marker is recovered can be used as a host to continue transforming a second target gene. A new engineered strain obtained after verifying the integration of the second target gene and the recovery of the screening marker can be used as a host for the conversion of other target genes, and so on, until target genes are all integrated into the genome and screening marker genes are removed. These genes were manipulated by molecular biology manipulations which is commonly applied in the field. The Yarrowia lipolytica CGMCC 28807 capable of efficiently and rapidly synthesizing erythritol can be obtained ultimately to overexpress the ten genes (or a total of 16 genes from SEQ ID No. 2 to SEQ ID No. 17).

A series of engineered strains of Yarrowia lipolytica are obtained by the molecular biology manipulations described above, including engineered strains overexpressing one or more of the ten genes described above, and preferably, the engineered strain is the Yarrowia lipolytica strain simultaneously overexpressing all the ten genes described above: Yarrowia lipolytica CGMCC No. 28807.

The construction method described in the present disclosure utilizes Yarrowia lipolytica strain (previously known as Candida lipolytica) capable of synthesizing erythritol as a chassis microorganism, by means of metabolic engineering and genetic engineering, an engineered Yarrowia lipolytica strain capable of synthesizing erythritol efficiently using glucose as a carbon source for fermentation. The method can significantly improve the ability to synthesize erythritol, including a significant reduction in fermentation time, an improved production efficiency, and an increased content of the target product erythritol in the fermentation broth, without the synthesis of by-products such as mannitol and arabitol.

In an embodiment of the present disclosure, an objective of employing a gene from Yarrowia lipolytica itself in step B1 is to avoid that the new strain constructed by the present disclosure falls into the category of transgenosis. Transgenesis refers to the transfer of genes from one organism into a genome of another different organism. In the present disclosure, any genes from organisms other than Yarrowia lipolytica's own genome are not involved, but only genes from Yarrowia lipolytica's own genome and genes related to the efficient synthesis of erythritol are combined for further enhanced expression in order to enhance the carbon flow to the synthesized product erythritol.

The above genes related to the synthesis of erythritol are expressed in Yarrowia lipolytica in the ways described above, and these ways are intended only as examples to illustrate how the target genes are integrated into Yarrowia lipolytica cells, rather than a limitation of the present disclosure.

In a fourth aspect, the present disclosure provides a use of the engineered yeast in the synthesis of erythritol.

In a fifth aspect, the present disclosure provides a method for synthesizing erythritol by fermentation of the engineered yeast, including the steps of:
S1. performing yeast fermentation in a fermentation medium, and performing isolation to obtain a fermentation broth and yeast cells containing erythritol, and
S2. isolating and purifying erythritol from the fermentation broth and yeast cells containing erythritol obtained in step S1.

In an embodiment of the present disclosure, the medium includes a carbon source, a nitrogen source, an inorganic salt, and water; and culture conditions include that: the fermentation culture is performed at an initial pH value of 3.0 to 7.0 and at a temperature of 25 to 35° C. by shaking or stirring.

Further, in some examples, the pH value includes 3.0 and 7.0.

Further, in some examples, the temperature includes 25° C., 28° C., 30° C., 32° C., 33° C. and 35° C.

Further, the carbon source in the medium includes glucose, an amount of the carbon source being 50-350 g/L.

Further, in some examples, an amount of the carbon source includes 50 g/L, 100 g/L, 200 g/L, 250 g/L, 300 g/L, 310 g/L, 320 g/L and 350 g/L.

Further, the nitrogen source in the medium includes a mixture of one or more of peptone, yeast extract powder, or yeast extract, dry powder of corn steep liquor, diammonium hydrogen phosphate, and ammonium citrate, a content of the nitrogen in the medium being 5-30 g/L.

In step S1, the inorganic salt in the medium includes one or more of magnesium sulfate, zinc chloride and ammonium citrate, a content of the inorganic salt in the medium being 0-1 g/L, and preferably being 0.1-0.5 g/L.

In an embodiment of the present disclosure, in step S1, during the fermentation culture, samples are taken at intervals to detect a remaining amount of the substrate carbon source and an amount of a product erythritol produced, and the fermentation is ended when the substrate carbon source is used up; or
when an amount of glucose remaining in the fermentation broth is less than 30 g/L, the amount of glucose is supplemented for continuous fermentation to further enhance the content of erythritol in the fermentation broth.

In an embodiment of the present disclosure, in step S2, the isolating and purifying includes the steps of: removing cells from medium to obtain a clarified erythritol-containing fermentation broth, performing concentration to obtain an erythritol-rich concentrated broth, performing primary crystallization to obtain a crude product of erythritol, re-dissolving of the crude product, removing ions by ion exchange, performing decoloration, performing concentration, performing secondary crystallization to obtain a refined product of erythritol, and drying the same.

Further, the yeast cells isolation includes the steps of: performing centrifugation or membrane filtration on the fermentation broth to remove yeast cells, adding water to wash the cells twice in order to fully recover the erythritol therein, and obtaining a clarified fermentation broth containing erythritol.

Compared with the prior art, the present disclosure has the following beneficial effects.

1) In the present disclosure, multiple GTPs (pathway I) are overexpressed, the glucose is transported into cells in a non-energy-consuming form rather than a phosphorylated form, and the efficiency of transporting the substrate carbon source, such as glucose, into the chassis cell is enhanced by utilizing synergy among multiple GTPs, significantly shortening the fermentation time of erythritol.

2) In the present disclosure, ETPs (pathway III) are introduced to avoid feedback inhibition caused by product erythritol accumulation, and the efficiency of product erythritol outputting extracellular is enhanced, thus efficiently synthesizing erythritol.

3) In the present disclosure, on the basis of pathways I and III, ETs, RPI, RPE, GLK, FPK, FBA, GFs, and GFDBTFs (pathway II) are further introduced, which greatly enhances the ability of intracellular erythritol synthesis, and further increases the efficiency of synthesizing product erythritol.

4) In the present disclosure, a global design strategy is employed, through the synergistic effect of three pathways, fermentation time for the synthesis of erythritol by the engineered yeast (CGMCC No. 28807) of the present disclosure under the same conditions is significantly reduced, from more than 80 h of a parental strain to less than 50 h, nearly 30 h reduced; the production efficiency of the synthesis of erythritol by fermentation is significantly increased, from 2.3 g/L·h of the parental strain to 4.6 g/L, a 100% improvement; and the yield of the synthesis of erythritol by fermentation is significantly increased, and it is able to conduct multiple consecutive feeding (glucose feeding), with the production of more than 350 g/L, at least 100 g/L·higher than that of the parental strain. These improvements have resulted in very beneficial implementation effects, and has remarkably practical value.

### BRIEF DESCRIPTION OF THE DRAWINGS

Other features, objectives and advantages of the present disclosure will become more apparent by reading the detailed description of the non-limited examples by reference to the following accompanying drawings:

FIG. 1 is a schematic diagram of the multi-pathway collaboration for efficient and rapid synthesis of erythritol;

FIG. 2 is a schematic diagram of the typical overexpression of a combination of DNA elements (expression cassette);

FIG. 3 is a schematic diagram of an overexpression vector (expression cassette) containing GTP genes;

FIG. 4 shows gene expression analysis of overexpressed GTPs1, GTPs2, GTPs3, and GTPs4 (GTPs1-4), where 1, 3, 5 and 7 are GTPs1-4 expressions of a comparative original strain, and 2, 4, 6 and 8 are GTPs1-4 expressions of an engineered strain CGMCC No. 28807;

FIG. 5 shows gene expression analysis of overexpressed ETs1, ETs2, ETs3, and ETs4 (ETs1-4), where 1, 3, 5 and 7 are ETs1-4 expressions of the parental strain, and 2, 4, 6 and 8 are ETs1-4 expressions of the engineered strain CGMCC No. 28807;

FIG. 6 shows gene expression analysis of overexpressed RPI, RPE, GLK, and ETP, where 1, 3, 5 and 7 are RPI, RPE, GLK, and ETP expressions of the parental strain, and 2, 4, 6 and 8 are RPI, RPE, GLK, and ETP expressions of the engineered strain CGMCC No. 28807; and

FIG. 7 shows gene expression analysis of overexpressed FPK, FBA, GFs and GFDBTFs, where 1, 3, 5 and 7 are FPK, FBA, GFs, and GFDBTF expressions of the parental strain, and 2, 4, 6 and 8 are FPK, FBA, GFs, and GFDBTF expressions of the engineered strain CGMCC No. 28807.

### DETAILED DESCRIPTION

The present disclosure is described in detail by reference to the accompanying drawings and specific examples below. The following examples are implemented on the premise of the technical solutions of the present disclosure, and detailed implementations and specific operation processes are provided, which will be helpful for those skilled in the art to further understand the present disclosure. It is to be noted that the scope of protection of the present disclosure is not limited to the following examples, and several adjustments and improvements made on the premise of the conception of the present disclosure belong to the scope of protection of the present disclosure.

### Example 1. Over-Expression of GTP Genes in Yarrowia lipolytica

A GTP expression cassette was synthesized in a sequence as shown in FIG. 2, specifically in the following sequence: upstream 26S rDNA sequence→promoter (hp4d)→GTP gene sequence→GTPs terminator sequence (Tcyc1)→loxP→promoter (P_{EXP})→mycophenolic acid screening marker (guaB)→terminator sequence (Taep)→loxP→downstream 26S rDNA sequence. A sequence of the synthesized expression cassette containing GTP genes is SEQ ID No. 18. The GTPs sequence selected in the example is SEQ ID No. 3, i.e. GTPs3. Other GTP genes were over-expressed in the same manner as in the example.

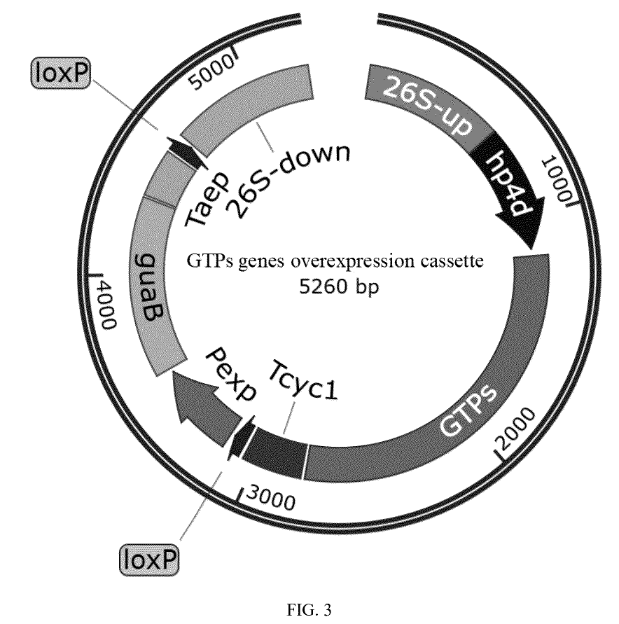

DNA elements in the constructed integration expression cassette containing GTP genes are shown in FIG. 3, the GTP genes are derived from Yarrowia lipolytica itself, with a gene sequence being one or more of SEQ ID No. 2-5. Screening markers could also be replaced with auxotrophic markers such as ura3 genes, or sucrase Suc2 genes, or with mycophenolic acid screening markers, and the mycophenolic acid screening marker was used in the example. The vector was used to transform Yarrowia lipolytica that synthesizes erythritol (e.g., Yarrowia lipolytica CGMCC No. 7326 or CGMCC No. 19351 previously obtained by the inventor, etc.), and screening was performed on a mycophenolic acid-containing medium. The screening medium is composed of: 6.7 g/L of YNB, 5 g/L of ammonium sulfate, 20 g/L of glucose, 300 mg/L of mycophenolic acid, 15 g/L of agar powder, and a pH value of 6.5. Since Yarrowia lipolytica is not inherently resistant to mycophenolic acid, a transformant that can grow in the mycophenolic acid-containing medium is able to grow and also contained GTP genes, and the transformant is a mutant expressing GTPs.

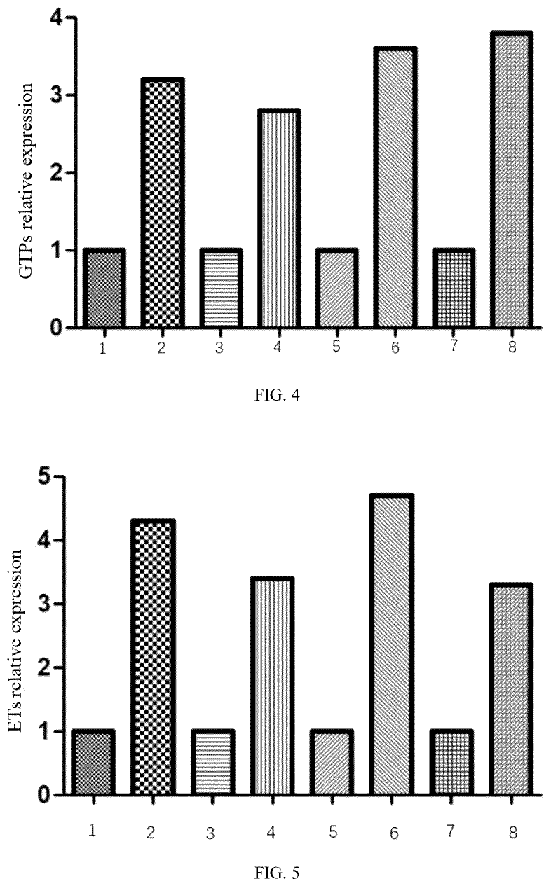

A plasmid pUB4-CRE containing Cre recombinase (from the published literature: Fickers et al. 2003. New disruption cassettes for rapid gene disruption and marker rescue in the yeast Yarrowia lipolytica. J. Microbiol. Methods, 55, 727-737) was transformed for the above-described mutant expressing GTPs, and screening was performed in a YPD agar medium containing hygromycin B as a selective marker (15 g/L of glucose, 5 g/L of yeast extract powder, 5 g/L of peptone, 15 g/L of agar, 350 μg/mL of hygromycin B, and a pH value of 6.5). The grown transformant was transferred in a minimal medium containing mycophenolic acid (6.7 g/L of YNB, 5 g/L of ammonium sulfate, 10 g/L of glucose, 300 mg/L of mycophenolic acid, 15 g/L of agar powder, and a pH value of 6.5) to select for mutants with lost mycophenolic acid maker. Mutants that could not utilize mycophenolic acid were then cultured in a liquid YPD medium without hygromycin B, and the mutants after cultivation were subjected to gradient dilution before being placed on a solid YPD medium without hygromycin B. Transformants were selected from the growing transformants to be transferred into a YPD agar medium containing hygromycin B, and mutants that could no longer be resistant to hygromycin B were selected, which were the overexpression of GTP genes. Mutants in which the mycophenolic acid marker gene was also lost were screened for, which could be used as hosts for overexpressing other genes (or for enhancing GTP genes). Total RNA extraction, reverse transcription and fluorescence quantification detection were performed according to the instructions of the fluorescence quantification detection kit from Nanjing Vazyme Biotech Co., Ltd (product number Q711-02/03, the kit name is ChamQ™ Universal SYBR®qPCR Master Mix). A total RNA of the mutant was extracted and subjected to reverse transcription, and a reverse transcription product was used as a template for fluorescence quantitative polymerase chain reaction (qPCR) to detect an expression level of the GTP genes, which was compared to an expression level of a comparative original strain Yarrowia lipolytica CGMCC No. 7326 or CGMCC No. 19351. It is found that an expression level of a messenger RNA (mRNA) of GTP genes of a new engineered strain is significantly increased over the comparative original strain (parental strain) (FIG. 4), indicating that the GTP genes are expressed in the new engineered strain. A transformant overexpressing GTP genes while losing screening markers was labeled as a mutant strain ery::GTPs3. The GTPs4 gene was transferred into ery::GTPs3 following the same manipulation method to obtain ery::GTPs3::GTPs4. FIG. 4 shows relative expressions of various genes transferred, where 1, 3, 5 and 7 show GTPs1-4 expressions of the parental strain, being set as 1, and 2, 4, 6 and 8 show GTPs1-4 expressions of the engineered strain Yarrowia lipolytica CGMCC No. 28807, showing that all the GTPs1-4 expressions of the engineered strain are significantly elevated over the comparative original strain, and indicating that the GTP genes are expressed in the engineered strain. Following the same genetic manipulation method, the GTPs1 and GTPs2 genes were further introduced into Yarrowia lipolytica to obtain a recombinant strain ery::GTPs1::GTPs2::GTPs3::GTPs4 overexpressing GTPs1::GTPs2, abbreviated as ery::GTPs.

The forward and reverse primer sequences for GTPs1, GTPs2, GTPs3 and GTPs4 gene expressions verification are shown in SEQ ID No. 19-20, 21-22, 23-24, 25-26, respectively.

The above-described mutants overexpressing GTP genes while losing mycophenolic acid screening marker genes were inoculated in a fermentation medium to conduct an experiment for the synthesis of erythritol, in order to compare with the comparative strain (CGMCC No. 19351). The fermentation medium is composed of 330 g/L of dextrose monohydrate, 8 g/L of yeast extract powder, 2 g/L of peptone, 3 g/L of ammonium citrate, 0.01 g/L of magnesium sulfate heptahydrate, 0.001 g/L of zinc chloride, and a pH value of 6.5, and was sterilized. Fermentation was carried out in a 3 L fermentation tank containing 1.5 L of fermentation medium at 30° C. by stirring at 700 rpm/min, with a ventilation rate of 1.2 vvm (volume per volume per minute). Periodic sampling and testing were performed, revealing that the mutant strain ery::GTPs3::GTPs4 consumed glucose completely in 70 h, with 185.5±3.5 g/L of erythritol synthesized without mannitol, and the efficiency of synthesizing erythritol of 2.64 g/L·h. In contrast, the comparative strain Yarrowia lipolytica CGMCC No. 19351 consumed all the glucose in 85 h, with 173±5.5 g/L of erythritol synthesized, and the efficiency of synthesizing erythritol of 2.0 g/L·h. Under the same fermentation conditions, the recombinant strain ery::GTPs consumed all the glucose in 68 h, with 189.5±2.5 g/L of erythritol synthesized without mannitol, and the efficiency of synthesizing erythritol of 2.74 g/L·h. From the fermentation results, it can be seen that by overexpressing GTP genes only, the efficiency of the synthesis of erythritol by Yarrowia lipolytica is improved from 2.0 to 2.64-2.74 g/L·h. The main reason for this is the elevated efficiency of glucose entering yeast cells, which can provide more carbon source for the synthesis of erythritol.

### Example 2. Over-Expression of ET Genes in Yarrowia lipolytica

The GTP genes (such as GTPs3) in the sequence SEQ ID No. 18 in Example 1 were replaced with ET genes (ETs1-4, SEQ ID No. 6-9), and the rest of DNA elements could be unchanged or changed accordingly, for example, other promoter sequences such as a glyceraldehyde-3-phosphate dehydrogenase gene promoter sequence could be used. In the example, only GTP genes were replaced, and a conversion method, a screening method and a fermentation method were the same as in Example 1.

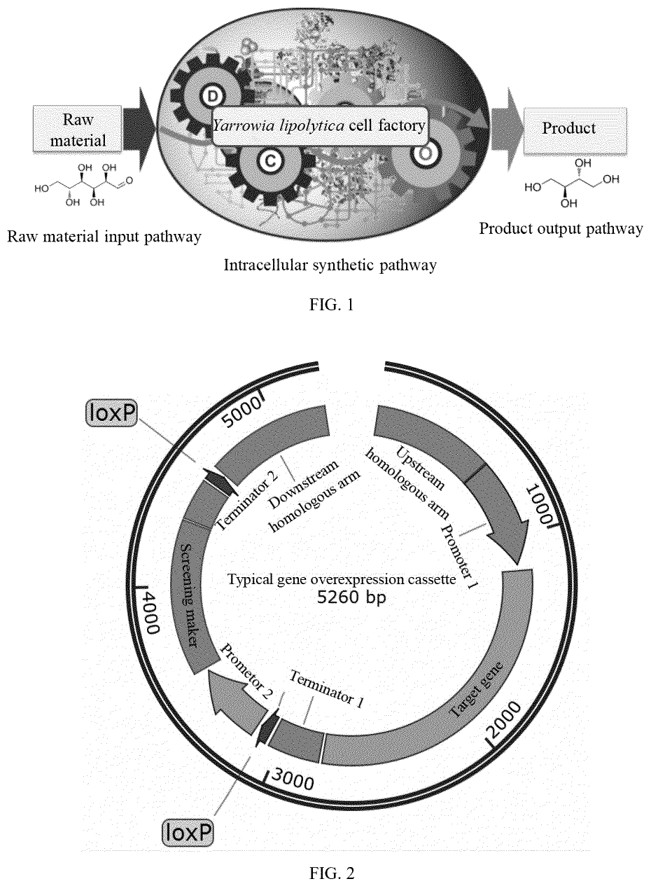

A transformant mutant strain overexpressing ET genes on the basis of strain overexpressing GTP genes was designated as ery::GTPs1::GTPs2::GTPs3::GTPs4::ETs1::ETs2::ETs3::ETs4 (abbreviated as ery::GTPs::ETs for ease of writing). Total RNA extraction, reverse transcription and fluorescence quantification detection were performed according to the instructions of the fluorescence quantification detection kit from Nanjing Vazyme Biotech Co., Ltd (product number Q711-02/03, the kit name is ChamQ™ Universal SYBR®qPCR Master Mix). A total RNA of overexpressed ET genes was extracted and subjected to reverse transcription, and a reverse transcription product was used as a template for qPCR to detect an expression level of ET genes, which was compared to a comparative strain Yarrowia lipolytica CGMCC No. 19351. It is found that an expression level of an mRNA of ET genes of the mutant strain is significantly increased over the comparative strain (FIG. 5), indicating that the ET genes are expressed in the mutant strain. In FIGS. 5, 1, 3, 5 and 7 represent ETs1-4 expressions of the comparative original strain, being set as 1, and 2, 4, 6 and 8 represent ETs1-4 expressions of an engineered strain Yarrowia lipolytica CGMCC.28807, showing that all the GTPs1-4 expressions of the engineered strain are significantly elevated over the comparative original strain, and indicating that ET genes are expressed in the engineered strain.

The forward and reverse primer sequences for verification of ETs1, ETs2, ETs3, and ETs4 genes expressions are shown in SEQ ID No. 27-28, 29-30, 31-32, 33-34.

The obtained transformant overexpressing ET genes was subjected to fermentation for the synthesis of erythritol, and the composition and conditions of the fermentation medium were the same as in Example 1. The effects of synthesis of erythritol by fermenting different strains are shown in Table 1 below.

TABLE 1

| | | | Efficiency of |
|---|---|---|---|
| | Production of | | synthesizing |
| | erythritol | Fermentation | erythritol |
| Strain | (g/L) | time (h) | (g/L · h) |
| ery::GTPs::ETs | 195 ± 3.5 | 60 ± 2 | 3.25 |
| Comparative | 172 ± 4.5 | 85 ± 3 | 2.0 |
| strain CGMCC | | | |
| No. 19351 | | | |

As shown in the above table, compared with the comparative strain, it is found that the simultaneous overexpression of GTP and ET genes can improve the efficiency of synthesizing erythritol more, about 15% higher than that of the mutant strain that only overexpressed GTPs.

### Example 3. Over-Expression of RPI Genes in Yarrowia lipolytica (SEQ ID No. 10)

The GTP genes in the sequence SEQ ID No. 18 in Example 1 were replaced with RPI genes, and the rest of DNA elements could be unchanged or changed accordingly, for example, other promoter sequences such as a TKL gene promoter sequence could be used. In the example, only GTP genes were replaced, and a conversion method, a screening method and a fermentation method were the same as in Example 1.

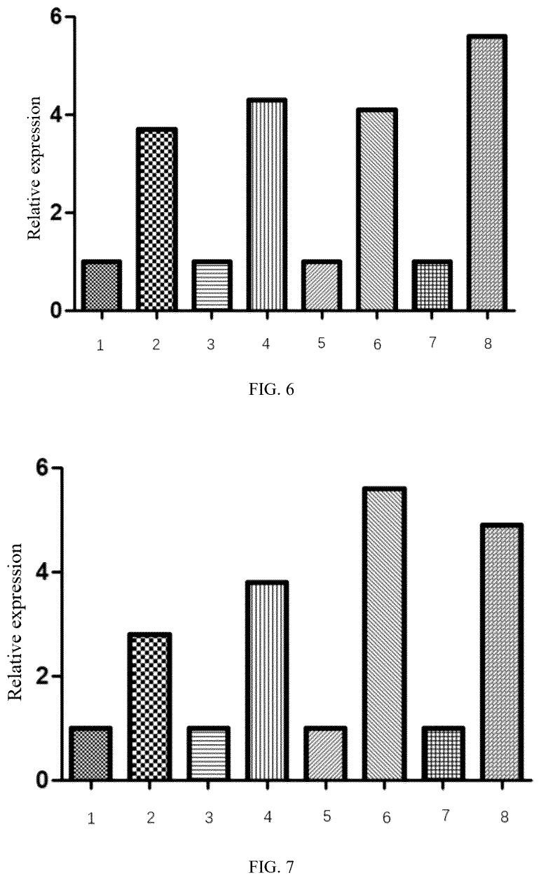

The methods of DNA conversion, screening of transformant strains and recycling of screening markers were the same as in Example 1. The chassis strain used could be the transformant ery::GTPs::ETs containing overexpressed ET genes obtained in Example 2, or other strains capable of synthesizing erythritol (such as the inventor's previously patented strain Yarrowia lipolytica CGMCC No. 7326, etc.). The obtained transformant containing RPI genes was designated as ery::GTPs::ETs::RPI, and the transformant overexpressing RPI genes only was designated as ery::RPI. Total RNA extraction, reverse transcription and fluorescence quantification detection were performed according to the instructions of the fluorescence quantification detection kit from Nanjing Vazyme Biotech Co., Ltd (product number Q711-02/03, the kit name is ChamQ™ Universal SYBR®qPCR Master Mix). A total RNA of the overexpressed RPI genes was extracted and subjected to reverse transcription, and a reverse transcription product was used as a template for qPCR to detect an expression level of RPI genes, which was compared to a comparative strain Yarrowia lipolytica CGMCC No. 19351. It is found that an expression level of an mRNA of RPI genes of the mutant strain is significantly increased over the comparative strain (FIG. 6), indicating that the RPI genes are expressed in the mutant strain. In FIGS. 6, 1, 3, 5 and 7 represent RPI, RPE, GLK and ETP expressions of the comparative original strain, being set as 1, and 2, 4, 6 and 8 represent RPI, RPE, GLK and ETP expressions of the engineered strain Yarrowia lipolytica CGMCC No. 28807, showing that all the RPI, RPE, GLK and ETP expressions of the engineered strain are significantly elevated over the comparative original strain, and indicating that the RPI, RPE, GLK and ETP are expressed in the engineered strain.

The forward and reverse primer sequences for verification of RPI, RPE, GLK and ETP genes expressions are shown in SEQ ID No. 35-36, 37-38, 39-40, 41-42.

The obtained transformant overexpressing RPI genes was subjected to fermentation to synthesize erythritol, and the composition and conditions of the fermentation medium were the same as in Example 1. The effects of the synthesis of erythritol and by-products are shown in Table 2.

TABLE 2

| | | | Efficiency of |
|---|---|---|---|
| | Production of | | synthesizing |
| | erythritol | Fermentation | erythritol |
| Strain | (g/L) | time (h) | (g/L · h) |
| ery::GTPs::ETs::RPI | 200 ± 3.5 | 58 ± 2 | 3.45 |
| ery::RPI | 175 ± 3.5 | 85 ± 3 | 2.0 |
| CGMCC No. 19351 | 174 ± 3.5 | 85 ± 3 | 2.0 |

As shown in the above table, compared with the comparative strain, it is found that the simultaneous overexpression of GTPs, ETs and RPI genes can further improve the efficiency of synthesizing erythritol, and the overexpression of RPI genes alone do not have a significant role, and other genes are required to be coordinated to play a positive function.

### Example 4. Over-Expression of RPE Genes in Yarrowia lipolytica

The GTP genes in the sequence SEQ ID No. 18 in Example 1 were replaced with RPE genes (SEQ ID No. 11), and the rest of DNA elements could be unchanged or changed accordingly, for example, other promoter sequences such as a TAL gene promoter sequence could be used. In the example, only GTP genes were replaced, and a conversion method, a screening method and a fermentation method were the same as in Example 1.

The methods of DNA conversion, screening of transformant strains and recycling of screening markers were the same as in Example 1. The chassis strain used could be the transformant ery::GTPs::ETs::RPI containing overexpressed ET genes obtained in Example 3, or other strains capable of synthesizing erythritol (such as the inventor's previously patented strain Yarrowia lipolytica CGMCC No. 7326, etc.). The strain obtained in Example 3 was used in the example. The obtained transformant containing RPE genes was designated as ery::GTPs::ETs::RPI:RPE, and the transformant overexpressing RPI genes only was designated as ery::RPI. Total RNA extraction, reverse transcription and fluorescence quantification detection were performed according to the instructions of the fluorescence quantification kit from Nanjing Vazyme Biotech Co., Ltd (product number Q711-02/03, the kit name is ChamQ™ Universal SYBR®qPCR Master Mix). A total RNA of the overexpressed RPE genes was extracted and subjected to reverse transcription, and a reverse transcription product was used as a template for qPCR to detect the expression level of RPE genes, which then was compared to a comparative strain Yarrowia lipolytica CGMCC No. 19351. It is found that an expression level of the mRNA of RPE genes of the mutant strain is significantly increased over comparative strain (FIG. 6), indicating that the RPE genes are expressed in the mutant strain.

The obtained transformant overexpressing RPE genes was subjected to fermentation to synthesize erythritol, and the composition and conditions of the fermentation medium were the same as in Example 1. The effects of the synthesis of erythritol and by-products are shown in Table 3.

TABLE 3

| | | | Efficiency of |
|---|---|---|---|
| | Production of | | synthesizing |
| | erythritol | Fermentation | erythritol |
| Strain | (g/L) | time (h) | (g/L · h) |
| ery::GTPs::ETs::RPI::RPE(ery::GERE) | 205 ± 3.5 | 56 ± 2 | 3.66 |
| ery::RPE | 176 ± 3.5 | 85 ± 3 | 2.0 |
| CGMCC No. 19351 | 174 ± 3.5 | 85 ± 3 | 2.0 |

As shown in the above table, compared with the comparative strain, it is found that the simultaneous overexpression of GTPs, ETs, RPI and RPE genes can further improve the efficiency of synthesizing erythritol. For ease of writing, Yarrowia lipolytica overexpressing GTPs, ETs, RPI and RPE simultaneously is designated as ery::GERE. There is no difference between the engineered strain overexpressing RPE alone and the comparative strain, indicating that it is necessary to coordinate with other genes to enhance the synthesis efficiency, reflecting the effect of synergistic function of multiple genes described in the present disclosure.

### Example 5. Over-Expression of Other Genes in Yarrowia lipolytica

Other genes include: GLK genes (SEQ ID No. 12), ETP genes (SEQ ID No. 13), FPK genes (SEQ ID No. 14), FBA genes (SEQ ID No. 15), GF genes (SEQ ID No. 16), and GFDBTFs (SEQ ID No. 17). The construction method, conversion method, and screening method for expression vectors containing these genes, and the method and conditions for fermentation of the transformant obtained were the same as those described in the above examples, which did not repeatedly describe here. The final engineered strain obtained overexpress the above ten genes of GTPs, ETs, RPI, RPE, GLK, ETP, FPK, FBA, GFs, GFDBTFs, with the genotype: ery::GTPs::ETs::RPI:RPE::GLK::ETP::FPK::FBA::GFs::GFDBTF, and for ease of writing, abbreviated as Yarrowia lipolytica ery989, and the accession number was Yarrowia lipolytica CGMCC No. 28807. The expression verification of various genes is shown in FIGS. 6 and 7, and expression levels of the corresponding genes of the engineered strain are obviously elevated after overexpression. In FIGS. 7, 1, 3, 5 and 7 represent FPK, FBA, GF and GFDBTF expressions of the comparative original strain, being set as 1, and 2, 4, 6 and 8 represent FPK, FBA, GF and GFDBTF expressions of the engineered strain Yarrowia lipolytica CGMCC No. 28807, showing that all the FPK, FBA, GF and GFDBTF gene expressions of the engineered strain are significantly elevated over the comparative original strain (CGMCC 19351), and indicating that the FPK, FBA, GF and GFDBTF genes are expressed in the engineered strain (CGMCC 28807).

The forward and reverse primer sequences for the verification of FPK, FBA, GFs and GFDBTF genes expressions are shown in SEQ ID No. 43-44, 45-46, 47-48, 49-50.

The obtained transformant ery989 was subjected to fermentation for synthesis of erythritol, and the conditions and composition of the fermentation medium were the same as in Example 1. The effects of the synthesis of erythritol are shown in Table 4.

TABLE 4

| | | | Efficiency of |
|---|---|---|---|
| | Production of | | synthesizing |
| | erythritol | Fermentation | erythritol |
| Strain | (g/L) | time (h) | (g/L · h) |
| Engineered strain ery989 | 215 ± 3.5 | 46 ± 2 | 4.67 |
| (CGMCC No. 28807) | | | |
| Comparative strain | 176 ± 3.5 | 85 ± 3 | 2.07 |
| CGMCC No. 19351 | | | |

As shown in the above table, compared with the comparative strain, it is found that the simultaneous overexpression of ten genes can further improve the efficiency of synthesizing erythritol, a 100% improvement in the synthesis efficiency (productivity), achieving a very significant implementation effect.

### Comparative Example 1

In the present disclosure, in order to demonstrate the implementation effect, a proprietary strain CGMCC No. 19351 from a previous patent filed by our laboratory (title: construction method for recombinant Yarrowia lipolytica for synthesizing erythritol and recombinant Yarrowia lipolytica strain, application number: 2020100692506.6) was taken as a chassis, and only ETP genes (SEQ ID No. 13) were transferred into the strain CGMCC No. 19351. The fermentation results showed that the obtained strain containing enhanced expression of SEQ ID No. 13 had a conversion rate (yield) of 60.8%, a fermentation time of 85 h, the production of 182.5 g/L, and a synthesis efficiency (productivity) of 2.14 g/L·h in synthesizing erythritol under the same conditions (as in Example 1). The implementation effect is far less than that of the engineered strain ery989 (CGMCC No. 28807), indicating that the yield has been improved by overexpressing ETP genes (SEQ ID No. 13) on the basis of strain CGMCC No. 19351. Multiple genes need to be coordinated for more dramatic effects. It is equivalent to the barrel theory that all short slabs rather than one or several short slabs require to be complemented to fill the barrel with water, achieving a desired effect.

### Comparative Example 2

A proprietary strain CGMCC No. 19351 from a previous patent (application number: 2020100692506.6) filed by our laboratory was taken as a chassis, and GTP genes (SEQ ID No. 2-5) and encoding genes of ETs, RPI, RPE, GLK, FPK, FBA, GFs, and GFDBTFs (SEQ ID No. 6-12, SEQ ID No. 14-17) were transferred into the strain CGMCC No. 19351, without additionally transferring ETP genes (SEQ ID No. 13) into the strain CGMCC No. 19351. Fermentation results of the obtained strain show that under the same conditions (fermentation conditions as in Example 1), a fermentation ending time was 55±2 h (when the carbon source glucose was completely consumed), longer than that of strain CGMCC No. 28807 (fermentation time less than 50 h), and a conversion rate (yield) was 67±1%, lower than that of strain CGMCC No. 28807 (≈71%), indicating that the synthetic performance of the strain requires the synergistic effect of multiple genes including ETP genes for better implementation.

### Comparative Example 3

A proprietary strain CGMCC No. 19351 from a patent (application number: 2020100692506.6) filed prior to this experiment was taken as a chassis, and ETP genes (SEQ ID No. 13) and ETs, RPI, RPE, GLK, FPK, FBA, GFs, and GFDBTF genes (SEQ ID No. 6-12, SEQ ID No. 14-17) were transferred into the strain CGMCC No. 19351, without additionally transferring GTP genes (SEQ ID No. 2-5) into the strain CGMCC No. 19351. The obtained strain was fermented under the same conditions as in Example 1, and a fermentation end time was 58±2 h (when the carbon source glucose was completely consumed), longer than that of strain CGMCC No. 28807 (fermentation time less than 50 h), and a conversion rate (yield) was 66±2%, lower than that of strain CGMCC No. 28807 (≈71%), indicating that the erythritol synthetic performance of the strain requires the synergistic effect of multiple genes including GTP genes for better implementation effect.

### Comparative Example 4

A proprietary strain CGMCC No. 19351 from a patent (application number: 2020100692506.6) filed by our laboratory was taken as a chassis, and only GTP genes (SEQ ID No. 2-5) and ETP genes (SEQ ID No. 13) were transferred into the strain CGMCC No. 19351, without additionally enhancing the expression of genes in ETs pathways. The obtained strain was fermented under the same conditions as in Example 1, and a fermentation ending time was 67±2 h, significant longer than that of strain CGMCC No. 28807 (fermentation time less than 50 h), and a production of erythritol was 188.5±2.5 g/L, lower than that of strain CGMCC No. 28807 (≈210 g/L), indicating that the transfer of transporter protein genes into the chassis strain alone cannot significantly enhance the synthesis performance of erythritol, and a variety of other genes are required to coordinate to further enhance erythritol synthesis.

### Comparative Example 5

The GTP genes in Example 1 were replaced with HK genes (see prior patent with application number of CN2020100692506.6 for the sequence), i.e., overexpression of HK genes again on the basis of strain CGMCC No. 19351 (note: the strain CGMCC No. 19351 had already overexpressed the HK genes, as detailed in the invention patent CN2020100692506.6). Conversion and fermentation methods were the same as in Example 1, and the fermentation results showed that the production of erythritol was 191 g/L for the strain overexpressing HK repeatedly, with a fermentation time of 78 h, which is not improved significantly compared to the comparative strain CGMCC No. 19351 with the production of 189 g/L and a fermentation time of 79 h, indicating that overexpression of HK alone do not lead to significant improvement of erythritol production.

The results from the above comparative examples show that the proprietary strain CGMCC No. 28807 obtained by the present disclosure has achieved extremely significant and even unexpected implementation effects because of the synergistic benefits of multiple genes of the present disclosure, and that the optimal implementation effect can be achieved by co-expression of the multiple genes of the present disclosure, which can shorten the fermentation time to less than 50 h, and improve the productivity (g/h-L) from 2.3 to 4.6, an increase of nearly 100%.

### Example 6. Optimization Experiment for Fermenting Yarrowia lipolytica Ery989 for Synthesis of Erythritol

A representative strain Yarrowia lipolytica ery989 for the best performance for synthesizing erythritol was selected for deposition, with an accession number of CGMCC No. 28807, and the multi-pathway collaboration for the efficient and rapid synthesis of erythritol by this strain was shown in FIG. 1. This representative strain was used as an example to carry out the optimization experiment of synthesis of erythritol by fermentation, including the following steps.

### (1) An Experiment of Biosynthesizing Erythritol by Fermenting Strain CGMCC No. 28807 at a Temperature of 25° C. and a Glucose Concentration of 50 g/L

A yeast strain CGMCC No. 28807 was inoculated in a 2 L baffled flask (with raised bottom edges to increase the effect of dissolved oxygen by stirring) containing 300 mL of fermentation medium for shaking at 220 rpm/min and at 25° C., with an initial cell density (OD₆₀₀) of 1.2, and the fermentation medium being composed of 50 g/L of glucose, 5 g/L of yeast extract powder, 2 g/L of peptone, 1 g/L of diammonium hydrogen phosphate, and an initial pH value of 6.5. Samples were taken at regular intervals to determine a glucose content and erythritol production. The glucose was consumed completely in 24 h, and the erythritol production was determined to be 25 g/L, with a conversion rate of 50% and a synthesis efficiency of 1.1 g/L·h, showing that a low initial glucose concentration leads to a low conversion rate and production efficiency. The reason may be that when the glucose concentration is low, most of the glucose is used for cell growth and less for the synthesis of product erythritol.

### (2) An Experiment of Biosynthesizing Erythritol by Fermenting Strain CGMCC No. 28807 at a Temperature of 25° C. and a Glucose Concentration of 200 g/L

A yeast strain CGMCC No. 28807 was inoculated in a 2 L baffled flask containing 200 mL of fermentation medium for shaking at 250 rpm/min and at 25° C., with an initial cell density (OD₆₀₀) of 1.2, and the fermentation medium being composed of 200 g/L of glucose, 6 g/L of yeast extract powder, 3 g/L of peptone, 2 g/L of diammonium hydrogen phosphate, 0.2 g/L of magnesium sulfate, 0.005 g/L of zinc chloride, and an initial pH value of 6.5. Samples were taken at regular intervals to determine a glucose content and erythritol production. The glucose was consumed completely in 38 h, and the erythritol production was determined to be 120 g/L, with a conversion rate of 60% and a synthesis efficiency of 3.15 g/L·h, showing that an increased glucose concentration leads to a significantly enhanced synthesis efficiency.

### (3) An Experiment of Biosynthesizing Erythritol by Fermenting Strain CGMCC No. 28807 at a Temperature of 28° C. and a Glucose Concentration of 300 g/L

A yeast strain CGMCC No. 28807 was inoculated in a 2 L baffled flask containing 200 mL of fermentation medium for shaking at 250 rpm/min and at 28° C., with an initial cell density (OD₆₀₀) of 1.2, and the fermentation medium being composed of 300 g/L of glucose, 8 g/L of yeast extract powder, 2 g/L of peptone, 3 g/L of ammonium citrate, 0.2 g/L of magnesium sulfate, 0.01 g/L of zinc chloride, and an initial pH value of 6.5. Samples were taken at regular intervals to determine a glucose content and erythritol production. The glucose was consumed completely in 48 h, and the erythritol production was determined to be 210 g/L, with a conversion rate of 70% and a synthesis efficiency of 4.37 g/L·h, showing that a further increased glucose concentration leads to a further enhanced synthesis efficiency.

### (4) An Experiment of Biosynthesizing Erythritol by Fermenting Strain CGMCC No. 28807 at a Temperature of 32° C. and a Glucose Concentration of 350 g/L

A yeast strain CGMCC No. 28807 was inoculated in a2 L baffled flask (with raised bottom edges to increase the effect of stirring) containing 200 mL of fermentation medium for shaking at 250 rpm/min and at 32° C., with an initial cell density (OD₆₀₀) of 1.2, and the fermentation medium being composed of 350 g/L of glucose, 10 g/L of yeast extract powder, 3 g/L of peptone, 4 g/L of diammonium hydrogen phosphate, 0.4 g/L of magnesium sulphate, and an initial pH value of 6.5. Samples were taken at regular intervals to determine a glucose content and erythritol production. The glucose was consumed completely in 55 h, and the erythritol production was determined to be 235 g/L, with a conversion rate of 67.1% and a synthesis efficiency of 4.27 g/L·h.

### (5) An Experiment of Biosynthesizing Erythritol by Fermenting Strain CGMCC No. 28807 at a Temperature of 33° C. and a Glucose Concentration of 320 g/L.

A yeast strain CGMCC No. 28807 was inoculated in a 2 L baffled flask (with raised bottom edges to increase the effect of stirring) containing 200 mL of fermentation medium for shaking at 250 rpm/min and at 33° C., with an initial cell density (OD₆₀₀) of 1.5, and the fermentation medium being composed of 320 g/L of glucose, 8 g/L of yeast extract powder, 3 g/L of peptone, 3 g/L of diammonium hydrogen phosphate, 0.4 g/L of magnesium sulphate, and an initial pH value of 5.5. Samples were taken at regular intervals to determine a glucose content and erythritol production. The glucose was consumed completely in 50 h, and the erythritol production was determined to be 218 g/L, with a conversion rate of 68.1% and a synthesis efficiency of 4.36 g/L·h.

### (6) An Experiment of Biosynthesizing Erythritol by Fermenting Strain CGMCC No. 28807 at a Temperature of 35° C. and a Glucose Concentration of 100 g/L

A yeast strain CGMCC No. 28807 was inoculated in a 2 L baffled flask (with raised bottom edges to increase the effect of stirring) containing 200 mL of fermentation medium for shaking at 250 rpm/min and at 35° C., with an initial cell density (OD₆₀₀) of 1.5, and the fermentation medium being composed of 100 g/L of glucose, 5 g/L of yeast extract powder, 2 g/L of peptone, 1 g/L of diammonium hydrogen phosphate, 0.05 g/L of magnesium sulphate, and an initial pH value of 6.5. Samples were taken at regular intervals to determine a glucose content and erythritol production. The glucose was consumed completely in 80 h, and the erythritol production was determined to be 45.5 g/L, with a conversion rate of 45.5% and a synthesis efficiency of 0.56 g/L·h. This is due to the heat intolerance of the strain, resulting in poor growth at a high temperature of 35° C., with an OD value of only 13, leading to a longer fermentation time and lower conversion rate.

### (7) An Experiment of Biosynthesizing Erythritol by Fermenting Strain CGMCC No. 28807 at a Temperature of 30° C., an Initial pH Value of 3.0 and a Glucose Concentration of 310 g/L

A yeast strain CGMCC No. 28807 was inoculated in a 2 L baffled flask (with raised bottom edges to increase the effect of stirring) containing 200 mL of fermentation medium for shaking at 250 rpm/min and at 30° C., with an initial cell density (OD₆₀₀) of 1.2, and the fermentation medium being composed of 310 g/L of glucose, 8 g/L of yeast extract powder, 3 g/L of peptone, 3 g/L of diammonium hydrogen phosphate, 0.2 g/L of magnesium sulphate, and an initial pH value of 3.0 adjusted by citric acid. Samples were taken at regular intervals to determine a glucose content and erythritol production. The glucose was consumed completely in 49 h, and the erythritol production was determined to be 213 g/L, with a conversion rate of 68.7% and a synthesis efficiency of 4.34 g/L·h.

### (8) An Experiment of Biosynthesizing Erythritol by Fermenting Strain CGMCC No. 28807 at a Temperature of 33° C. and a Glucose Concentration of 250 g/L

A yeast strain CGMCC No. 28807 was inoculated in a 2 L baffled flask (with raised bottom edges to increase the effect of stirring) containing 200 mL of fermentation medium for shaking at 250 rpm/min and at 33° C., with an initial cell density (OD₆₀₀) of 1.2, and the fermentation medium being composed of 250 g/L of glucose, 10 g/L of yeast extract, 2 g/L of dry powder of corn steep liquor, 3 g/L of diammonium hydrogen phosphate, 0.2 g/L of magnesium sulphate, and an initial pH value of 5.5. Samples were taken at regular intervals to determine a glucose content and erythritol production. The glucose was consumed completely in 52 h, and the erythritol production was determined to be 155 g/L, with a conversion rate of 62% and a synthesis efficiency of 2.98 g/L·h.

### (9) An Experiment of Biosynthesizing Erythritol by Fermenting Strain CGMCC No. 28807 at a Temperature of 30° C., an Initial pH Value of 7.0 and a Glucose Concentration of 300 g/L

A yeast strain CGMCC No. 28807 was inoculated in a 2 L baffled flask (with raised bottom edges to increase the effect of stirring) containing 200 mL of fermentation medium for shaking at 250 rpm/min and at 30° C., with an initial cell density (OD₆₀₀) of 1.2, and the fermentation medium being composed of 300 g/L of glucose, 8 g/L of yeast extract powder, 2 g/L of peptone, 3 g/L of diammonium hydrogen phosphate, 0.2 g/L of magnesium sulphate, and an initial pH value of 7.0 adjusted by sodium hydroxide. Samples were taken at regular intervals to determine a glucose content and erythritol production. The glucose was consumed completely in 48 h, and the erythritol production was determined to be 215 g/L, with a conversion rate of 71.6% and a synthesis efficiency of 4.47 g/L·h.

In each of the above embodiments of fermentation, during the fermentation, the evaporated water was to be replenished regularly to the weight at which the fermentation was initiated. The weight of the fermentation flask containing fermentation broth was noted at the start of fermentation, and the weight was noted again at each sampling, and sterile water was used to replenish the water to the weight at the start of fermentation. The volume of each sampling was 0.2 mL, and the sample, after being diluted 10-20 times, was used for high performance liquid chromatography (HPLC) to detect the content of carbon source (such as glucose) and erythritol production. An analytical column is a Shodex SP0810 sugar column at temperature of 70° C., a refractive index detector is used, and the pure water as mobile phase at a flow rate of 1 mL/min.

### (10) An Experiment of Biosynthesizing Erythritol by Fermenting Strain CGMCC No. 28807 in a 5 L Fermentation Tank

A yeast strain CGMCC No. 28807 was inoculated in a 5 L fermentation tank containing 3 L of fermentation medium for fermentation at 30° C. by stirring at an initial speed of 500 rpm/min, with an initial cell density (OD₆₀₀) of 1.2, the fermentation medium being composed of 310 g/L of glucose, 6 g/L of yeast extract powder, 2 g/L of peptone, 3 g/L of ammonium citrate, 2 g/L of diammonium hydrogen phosphate, 0.05 g/L of magnesium sulphate, and an initial pH value of 6.5, a ventilation rate of 3 L/min, and the stirring speed being increased to 700 rpm/min and the ventilation volume being increased to 5 L/min when OD₆₀₀ of the cell density was greater than 10.0. Samples were taken at regular intervals to determine a glucose content and erythritol production. The glucose was consumed completely in 46 h, and the erythritol production was determined to be 214.6 g/L, with a conversion rate of 69.2% and a synthesis efficiency of 4.66 g/L·h.

### (11) An Experiment of Biosynthesizing Erythritol by Fermenting Strain CGMCC No. 28807 in a 200 L Fermentation Tank

A yeast CGMCC No. 28807 strain was inoculated in a 200 L fermentation tank containing 140 L of fermentation medium for fermenting at 30° C. by stirring at an initial speed of 500 rpm/min, with an initial cell density (OD₆₀₀) of 1.2, the fermentation medium being composed of 310 g/L of glucose, 6 g/L of yeast extract powder, 2 g/L of peptone, 3 g/L of ammonium citrate, 2 g/L of diammonium hydrogen phosphate, 0.05 g/L of magnesium sulfate, and an initial pH value of 6.5, a ventilation volume of 140 L/min, and the stirring speed being increased to 600 rpm/min and the ventilation volume being increased to 200 L/min when OD₆₀₀ was over 10.0. Samples were taken at regular intervals to determine a glucose content and erythritol production. The glucose was consumed completely in 47 h, and the erythritol production was determined to be 217.8 g/L, with a conversion rate of 70.2% and a synthesis efficiency of 4.63 g/L·h.

### (12) An Experiment of Biosynthesizing Erythritol by Fed-Batch Fermentation of Strain CGMCC No. 28807 in a 200 L Fermentation Tank

A yeast strain CGMCC No. 28807 was inoculated in a 200 L fermentation tank containing 110 L of fermentation medium for fermenting at 30° C. by stirring at an initial speed of 500 rpm/min, with an initial bacterial density (OD₆₀₀) of 1.2, the fermentation medium being composed of 300 g/L of glucose, 8 g/L of yeast extract powder, 3 g/L of peptone, 3 g/L of ammonium citrate, 3 g/L of diammonium hydrogen phosphate, 0.05 g/L of magnesium sulfate, and an initial pH value of 6.5, a ventilation volume of 200 L/min, and the stirring speed being increased to 600 rpm/min and the ventilation volume being increased to 250 L/min when OD₆₀₀ was over 5.0. Samples were taken at regular intervals to determine a glucose content and erythritol production. When the glucose content was at 50 g/L, 10 L of 600 g/L sterilized glucose solution was added for continuous fermentation, with a total of 3 times of feeding. The fed glucose was consumed completely in 82 h, and the erythritol production was determined to be 353.6 g/L, with a synthesis efficiency of 4.31 g/L·h. It can be seen that the glucose feeding is beneficial to the increase in the concentration of erythritol, can save the hot steam cost for erythritol concentration, and has a significantly beneficial effect.

In each of the above examples of fermentation, all the fermentation media were sterilized and cooled to room temperature before being inoculated with strain.

(13) An Experiment of Purification of Erythritol from a Fermentation Broth

At the end of fermentation, the fermentation broth was loaded into a 1000 mL centrifuge tube and then centrifuged at 8000 g for 10 min to obtain clarified supernatant containing erythritol. Yeast cells were precipitated before being washed in suspension with 200 mL of purified water to release intracellular erythritol, and centrifugation was performed to obtain supernatant again. The fermentation supernatant and the solution from the washed cells were combined before being transferred into a rotary evaporation flask for evaporation and concentration, during which soluble solid content was measured, and the evaporation was stopped when the soluble solid content reached 66%. The concentrated solution was transferred into a flask, the flask containing the concentrated solution was placed in a gradient cooler and stirring was performed slowly with a magnetic stirring bar at 55 rpm/min. When the temperature dropped to 30° C., a seed crystal was added, leaving the flask for standing, and visible fine granular crystals appeared. As the temperature decreased gradually, the amount of crystallization gradually increased, and at this time the stirring speed was increased to 80 rpm/min. When the amount of crystallization no longer increased, the stirring was stopped. Centrifugal separation of crystals was performed to obtain a crude product of erythritol, which was re-dissolved until the soluble solid content reached 50%. Ion exchange, decolorization, removal of ions and pigments were performed in sequence before concentration, crystallization, centrifugation and drying to obtain a white refined product of erythritol.

The new engineered strain Yarrowia lipolytica CGMCC No. 28807 obtained in the present disclosure produced 214.6 g/L erythritol from 310 g/L glucose under optimized fermentation conditions in a 5 L fermentation tank in 46 h, with a conversion rate of 69.2% and a production efficiency of 4.66 g/L·h. 217.8 g/L erythritol was produced from 310 g/L glucose in 47 h in a 200 L fermentation tank, with a conversion rate of 70.2% and a production efficiency of 4.63 g/L·h, which were essentially the same as that of the 5 L fermentation tank. After three continuous feeding of glucose under fed-batch fermentation conditions in a 200 L fermentation tank, the fermentation finally ended after 82 h, with the production of erythritol reaching 353.6 g/L, the highest yield reported so far, and the production efficiency reaching 4.31 g/L·h.

The technology provided by the present disclosure had significant and unexpected implementation effects compared to the technology described in other publicly reported literature, and in particular, the production efficiency reached a maximum of 4.66 g/L·h, increasing nearly 100% compared to what had been reported in the literature, and the total production of 350 g/L was reached, the highest reported production to date.

In particular, it is to be noted that the selective overexpression of various genes in the present disclosure is not arbitrarily selected, but has been verified by repeated experiments and has achieved the above-described beneficial effects. Although specific examples of the present disclosure are described above, it is to be understood that the present disclosure is not limited to the particular embodiment described above, and the inventors can make various modifications within the scope of the claims, which do not affect the implement or application of the present disclosure.

1. An engineered yeast for efficient and rapid synthesis of erythritol, obtained by introducing genes related to the synthesis of erythritol by using a Yarrowia lipolytica strain as a chassis microorganism.

2. The engineered yeast according to claim 1, wherein the Yarrowia lipolytica strain has a genome containing a deoxyribonucleic acid (DNA) sequence having 97% or more homology or similarity to a sequence of SEQ ID No. 1 and is capable of synthesizing erythritol.

3. The engineered yeast according to claim 1, wherein the Yarrowia lipolytica strain comprises any one of Yarrowia lipolytica CGMCC 7326, Yarrowia lipolytica ery929 CGMCC No. 18478, and Yarrowia lipolytica ery929 CGMCC No. 19351.

4. The engineered yeast according to claim 1, wherein the genes related to the synthesis of erythritol comprise one or more of the following genes:
(1) genes glucose transporter proteins (GTPs) 1, GTPs2, GTPs3 and GTPs4 encoding GTPs as shown in SEQ ID No. 2-5;
(2) genes erythritol synthases (ETs)1, ETs2, ETs3 and ETs 4 encoding ETs as shown in SEQ ID No. 6-9;
(3) genes encoding ribulose-5-P isomerase (RPI) as shown in SEQ ID No. 10;
(4) genes encoding ribulose-5-P epimerase (RPE) as shown in SEQ ID No. 11;
(5) genes encoding glucose kinase (GLK) as shown in SEQ ID No. 12;
(6) genes encoding erythritol transporter proteins (ETPs) as shown in SEQ ID No. 13;
(7) genes encoding fructose-6-P kinase (FPK) as shown in SEQ ID No. 14;
(8) genes encoding fructose-1,6-bisphosphate aldolase (FBA) as shown in SEQ ID No. 15;
(9) genes encoding growth factors (GFs) as shown in SEQ ID No. 16; and
(10) genes encoding growth factor-DNA-binding transcription factors (GFDBTFs) as shown in SEQ ID No. 17.

5. The engineered yeast according to claim 1, wherein the engineered yeast is a Yarrowia lipolytica strain with a deposition number of CGMCC No. 28807.

6. A construction method for an engineered yeast for efficient and rapid synthesis of erythritol according to claim 1, comprising the following steps:
A1. designing a gene expression cassette for efficient synthesis of erythritol and synthesizing the gene expression cassette,
A2. transforming the gene expression cassette synthesized in step A1 into Yarrowia lipolytica, and
A3. screening for Yarrowia lipolytica containing the gene expression cassette;
or,
B1. combining a gene for efficient synthesis of erythritol as shown in any one or more of SEQ ID No. 2 to SEQ ID No. 17 with a promoter and a terminator of the gene to form a gene open reading frame (ORF), and
B2. screening for Yarrowia lipolytica containing the gene ORF.

7. A construction method for an engineered yeast for efficient and rapid synthesis of erythritol according to claim 2 comprising the following steps:
A1. designing a gene expression cassette for efficient synthesis of erythritol and synthesizing the gene expression cassette,
A2. transforming the gene expression cassette synthesized in step A1 into Yarrowia lipolytica, and
A3. screening for Yarrowia lipolytica containing the gene expression cassette;
or,
B1. combining a gene for efficient synthesis of erythritol as shown in any one or more of SEQ ID No. 2 to SEQ ID No. 17 with a promoter and a terminator of the gene to form a gene open reading frame (ORF), and
B2. screening for Yarrowia lipolytica containing the gene ORF.

8. A construction method for an engineered yeast for efficient and rapid synthesis of erythritol according to claim 3 comprising the following steps:
A1. designing a gene expression cassette for efficient synthesis of erythritol and synthesizing the gene expression cassette,
A2. transforming the gene expression cassette synthesized in step A1 into Yarrowia lipolytica, and
A3. screening for Yarrowia lipolytica containing the gene expression cassette;
or,
B1. combining a gene for efficient synthesis of erythritol as shown in any one or more of SEQ ID No. 2 to SEQ ID No. 17 with a promoter and a terminator of the gene to form a gene open reading frame (ORF), and
B2. screening for Yarrowia lipolytica containing the gene ORF.

9. A construction method for an engineered yeast for efficient and rapid synthesis of erythritol according to claim 4 comprising the following steps:
A1. designing a gene expression cassette for efficient synthesis of erythritol and synthesizing the gene expression cassette,
A2. transforming the gene expression cassette synthesized in step A1 into Yarrowia lipolytica, and
A3. screening for Yarrowia lipolytica containing the gene expression cassette;
or,
B1. combining a gene for efficient synthesis of erythritol as shown in any one or more of SEQ ID No. 2 to SEQ ID No. 17 with a promoter and a terminator of the gene to form a gene open reading frame (ORF), and
B2. screening for Yarrowia lipolytica containing the gene ORF.

10. A construction method for an engineered yeast for efficient and rapid synthesis of erythritol according to claim 5 comprising the following steps:
A1. designing a gene expression cassette for efficient synthesis of erythritol and synthesizing the gene expression cassette,
A2. transforming the gene expression cassette synthesized in step A1 into Yarrowia lipolytica, and
A3. screening for Yarrowia lipolytica containing the gene expression cassette;
or,
B1. combining a gene for efficient synthesis of erythritol as shown in any one or more of SEQ ID No. 2 to SEQ ID No. 17 with a promoter and a terminator of the gene to form a gene open reading frame (ORF), and
B2. screening for Yarrowia lipolytica containing the gene ORF.

11. The construction method according to claim 6, wherein in step A1, the gene expression cassette for the synthesis of erythritol comprises an upstream homologous integration arm sequence, a downstream homologous integration arm sequence, a promoter sequence, a terminator sequence, a screening marker sequence, and a sequence of the genes related to the synthesis of erythritol as shown in any one or more of SEQ ID No. 2 to SEQ ID No. 17.

12. A use of an engineered yeast according to claim 1 in the synthesis of erythritol.

13. A use of an engineered yeast according to claim 2 in the synthesis of erythritol.

14. A use of an engineered yeast according to claim 3 in the synthesis of erythritol.

15. A use of an engineered yeast according to claim 4 in the synthesis of erythritol.

16. A use of an engineered yeast according to claim 5 in the synthesis of erythritol.

17. A use of an engineered yeast obtained by a construction method according to claim 5 in the synthesis of erythritol.

18. A use of an engineered yeast obtained by a construction method according to any one of claim 6 in the synthesis of erythritol.

19. A method for synthesizing erythritol by fermenting an engineered yeast according to claim 1 comprising the following steps:
S1. performing yeast fermentation in a fermentation medium, and performing isolation to obtain a fermentation broth and yeast cells containing erythritol, and
S2. isolating and purifying erythritol from the fermentation broth and yeast cells containing erythritol obtained in step S1.

20. The method according to claim 19, wherein in step S1, the medium comprises a carbon source, a nitrogen source, an inorganic salt, and water, wherein the carbon source in the medium comprises glucose, an amount of the carbon source being 50-350 g/L, the nitrogen source in the medium comprises a mixture of one or more of peptone, yeast extract powder, or yeast extract, dry powder of corn steep liquor, diammonium hydrogen phosphate, and ammonium citrate, a content of the nitrogen source in the medium being 5-30 g/L, and the inorganic salt in the medium comprises one or more of magnesium sulfate, zinc chloride, and ammonium citrate, a content of the inorganic salt in the medium being 0-1 g/L; and culture conditions comprise that the fermentation culture is performed by shaking or stirring at an initial pH value of 3.0-7.0 and a temperature of 25-35° C.


SEQUENCE LISTING (50 sequences)

SEQ ID NO: 1 - DNA, 1397 bp, synthetic construct
  source 1..1397: mol_type = other DNA
cagatcttggtggtagtagcaaatattcaaatgagaactttgaagactgaagtggggaaaggttccgtgtgaacagcagttggacacgggtaagtcgatcctaaggggtggcataactgtcgcgtacggcccgataagggccttctccaaaagggaagccggttgaaattccggcacttggatgtggattctccacggcaacgtaactgaatgtggggacggtggcacaagtcttggaaggagttatcttttctttttaacggagtcaacaccctggaattagtttgtctagagatagggtatcgttccggaagaggggggcagctttgtcccctccgatgcacttgtgacgccccttgaaaacccgcaggaaggaatagttttcacgccaagtcgtactgataaccgcagcaggtctccaaggtgaacagcctctagttgatagaataatgtagataagggaagtcggcaaaatagatccgtaacttcgggataaggattggctctgggggttggtggatggaagcgtgggagaccccaagggactggcagctgggcaactggcagccggacccgcggcagacactgcgtcgctccgtccacatcatcaaccgccccagaactggtacggacaaggggaatctgactgtctaattaaaacatagctttgcgatggttgtaaaacaatgttgacgcaaagtgatttctgcccagtgctctgaatgtcaaagtgaagaaattcaaccaagcgcgcgggtaaacggcgggagtaactatgctctcttaaggtagccaaatgcctcgtcatctaattagtgacgcgcatgaatggattaacgagattcccactgtccctatctactatgtagcgaaaccacagccaagggaacgggcttggcagaatcagcggggaaagaagaccctgttgagcttgactctagtttgacattgtgaagagacatagggggtgtagaataagtgggagcttcggcgccggtgaaataccactacccttatcgtttctttacttatttagtaagtggaagtggtttaacaaccattttctagcattcctttccaggctgaagacattgtcaggtggggagtttggctggggcggcacatctgttaaaagataacgcagatgtcctaagggggactcaatgagaacagaaatctcatgtagaacaaaagggtaaaagtccccttgattttgattttcagtgtgaatacaaaccatgaaagtgtggcctatcgatcctttagttgttcggagtttgaacctagaggtgccagaaaagttaccacagggataactggcttgtggcagtcaagcgttcatagcgacattgctttttgatccttcgatgtcggctcttcctatcataccgaagcag

SEQ ID NO: 2 - DNA, 1548 bp, synthetic construct
  source 1..1548: mol_type = other DNA
atgggactcgctaacatcatcaaccgtggagaaaagcccgagggctcggccttcatggcggcctttgtggccgtgtttgtcgcgtttggaggtattctgtttggatacgacactggaaccatttccggcgtcatggccatgccattcgtcaagaagacctttacagatgacggcctggagttcacttctgagcagacctcgctcatcacttccattctttctgcaggcaccttcactggagccatttctgctccctgggcctctgatactctgggaagacgactgggtctgatcctcttctgtgtcgtcttctctgttggcgctattcttcagactgctgccaccggccgaacgcttttgattgtcggacgagttgttgctggtcttggtgttggtggagtctcttccatcgttcctctttaccagtctgaggttgcccccaagtggatccgaggtgccgttgtctccatctaccagtttgccatcaccattggtcttctgctggctgccattgtcaacaacgcaaccaaaaacaaagacaacagtgcttcctaccgaattcctctcggccttcagcttctgtgggccgtcatcctgagtggaggtctcatcctgctaccggagactcctcgattctggatcaagaagggcgagtacgacaaggccgccgattccctgcgacgactacgacgacttcctgttgagcacgaggctgtacagaaggagctcctggagatccaatcttctcacgaccacgagatgcagatcggtagcgccacctgggccgcctgcttctcccccaaggggtcccagctgaagcgaatgctgaccggtattgccattcaggccctgcagcagctcaccggtatcaacttcatcttctactacggaaccgagttcttcaagaagtccaacatctccaaccccttcctcatccagatgatcaccaacattgtcaacgtggttatgaccatccccggtatcatgtttgttgatcgagtcggacgacgaaagctgctgttgatcggagctatcgtcatgtgctcttccgagtttatcgtggcggctgttggtactgccattgataacgagacctcctcaaaggttctgattgccttcacttgtaccttcattgccggtttcgccgccacctggggtcctattgcctgggttgtcattggagagattttccctctacgaatccgagccaagggtgttgctctatgcgccgcctccaactggcttttcaactttgccattgcctttgcaaccccctacctcgtcgacgaggcccctggatcggccggtctcaagaccaaggtcttcttcatctggggaggctgcaacttcctgtgcatcgccttcacttacttcttcatctacgagaccaagggtcttactctggaggaggtggaccagatgtacgccgagatcaagattgcttctcgatcccaccagtttgtgcctaccactcgagtcgctgcttacgacgagcacgcttctgacgacaagaaggacggacagcacgtctacattgagtctgtctag

SEQ ID NO: 3 - DNA, 1602 bp, synthetic construct
  source 1..1602: mol_type = other DNA
atgtccactagtgctatgaccgacgattatcccatggctgaggtctccattactgaggagaccaagatgcctgagcctgccaagaaatcgcagcttcttgtcattctgctctgtctctttgccgccatgggcggttttgtctttggctatgacactggtaccatttctggcttcatcaacatggaccctttcctagagagctttggagaagccgatgagtctggcgttttctttctatcccgaatccgagctgggctcattgttggactcttttccattggtgctttccttggcacccttcttggtggcttccttgctgacaaggttggtcgaaagaagggaattgtctatgtcgccatggtctacattgttggtatgcttatccagatcacagccttcacagcctggtaccagattgccattggtcgtgtcatcggtggtgttggtattggtgcactctctgtgctggtccccatgttccagtcagagactgctccccagaatcttcgaggcgctcttgtctcttctttccagctgtttattactctcggcatcttcattggatttagtgtttgctacgctaccaagtcgcggctcgataccggtgcctaccgaatccccatgggtctttgcttcgcctgggcttttatccttctcattggcatgtgcttcatgccagagtcccctcgattcctggtttcaattggccgcattgaggaggcccgaaaggctatggccatgacaaaccaggttcctctccacgacgctgtcattgatgaggaactcaaggctattgagaactctgtcattagggaaaagagtgctggtaaggccacatggaaagaacttttcactggcgagccccgaatgggctaccggcttacccttggtattctcgtccaggttctccagcagctctgtggtgccaactactttttctattacggaacttctatcttcaaggccattggaatgtcggattctttcgctacttcgatgatctttggcggaatcaacctcctgtctacctttggaggtctctacattgttgatcgattcggtcgacgaaagtgccttcttggtggagccatggtcatgtttgtctgtttcctcgtctattctactgtcgggttcgctgctctttatcccaacggtgataccacccttcccgccagaaaggctgttggtgatgtcatgattctgttcacctgtatcttcatcgccgctttcgcctctacgtgggcccctattgccttcgtcgttgtttctgagaccttccctctcagaatgcgatcaaagggaatggctgttgctactggaggaaactggatgatcaacttcctcgtttctttcctgactcccttcatcaccagttccattgggttcaagtacggttatgtcttcactgcttgcattggtttcgccattatctttgtcttcttcttcatccctgagaccaagggtctgtcccttgaggatgttgacgagctgtacgcttcaggtgtcagtgctcgaaactcccccaactgggttccttcttccaagaagcaggaagtcaacgaacctaagaacagtatgaccgacttctccgagtcctcttag

SEQ ID NO: 4 - DNA, 2446 bp, synthetic construct
  source 1..2446: mol_type = other DNA
atggcgaggctttgtctttctcaaacgggctcgattcgaagccggcgacgactctgggccggggtcggaacaatagccgactttcacactccagatccgccccacattttccatgcgtgcgcttgcacaaaacgctctcatatgcgtatgttggggaacatgacgcgcttagttgcacgtcatagaagacggcctatagcgtctatagtatggcaaatgatgacggggacttgaccgtcgcaccaagaaagccgtcatcaactcataccacaacagtgtgcccccctccacttacctcggcccacatgtcttaccactactccttccacatccagagctcacatggttatagcacggcacaagccctctaggccccaaggtttggagctgtgaagccctcttttggattcgcatgcttgaccaatgtaaacttcctctgccccgccactcctcgaaccatctcgtcaactctctggtggagttgggactcgcaggacaaacatgtccctgacacacaatacaagaccctgacacaaggcacaagacctgggccgttacaccggcccaccgccaagctcatcgcaacacaaagacgcgcgctggagtcgatgggctcttgacaggctccggaaactcagctgccgctcagagtttagttgcggcgagagttgcagagttggagtctgtttgaaaacgaaaagtgcaaggccaacgagcagcagtctcttgttccaaacacggatcggagtagcaccaaacacttataaataacgcgtccccagattccccagattctctttccactttgttctccaccaacttcttgtacacctctcaactcacaatgtacaaggtccacaacccctaccttacggcggcggtagccaccatgggcggtatgctctttggtttcgatatctcgtcggtttccgcctttgtcggcgaagaccagtacatgcagtactttggtcaccccacctccatccagcagggaggtatcacagcctccatggccggcggctccatgctgtcgtgttcgtttgccggtttcatttctgaccgaatcggccgaaagcccactatccagattgctgctgcctggtggatagtcggcgcggccattcagtgctcagcccagaacatgggccagctgattgccggccgtgtcatctccggtattggtattggtattggttcttctcagattcctgtcttcatttccgagctgtcccccaagaagattcgaggtcggctggtcggctgtttccagtgggccgtcacctggggtattctgatcatgttctacatctcctttggttgctcgtacatcaagggccctgcttctttccgtctggcctggggtatccagatgattcccggagccatgctggcgtttggaatgatgttcctggacgagtcgcctcgatggctggcatccaaggaccgttgggaagaggccatccagatcatccgatctgtcaacggcaactacggtaccgaggaggacattcttatggagattgaagacttgcgagaagtggtgcgaatcgaccacgagtccaagaccgtcatggtgtgggatctgttccgaaaggactccatcaaccgaaccatggtgggtatctgggcccagatctggcagcagctcaccggaatgaacgtcatgatgtactacgtggtcatcatcttcaagatggccggctactccggaaagtcttctgttcttgtctcctcctctatccagtacattatcaacgtggtcatgaccattcctgccctgctcttcattgataagattggacgacgacctctgctcattgtcggatgtctcttcatgatgacctggctgtttgccgtcggaggaatgctcggtgcttacggtacccagatgcccggaggtctgcctgccaaccccgaaaagaacattgctgccgacccctacaccaccatcttcatcgacgacaaccacaagactgcgcgaaaggccatcattgcctgctgctatctgtttgtggcctcgtttgcgcccacctggggtcccggtatctggctctactgttcggaaatcttccccaacaagcagcgagctatggccaactccatcaccgctggagccaactgggctttcaactttgctctggccatgtttgtgcctaccgccttcaagaacatcaactggaagacgtacatcatcttcggtgtcttctgtgtcgtcatgtgcatccacgtcttcctgctcttccccgagaccaagggtaagactctggaggagattgacatgatgtgggacgccaaggtgcctgcgtggcgaaccgcctcctgggtgcccgaccacatgccttctcgatacgccgagaaggatctggacgagaagcccatggacgagcttgctgaggagccttctcacgaggaggaccctcatcctcagtacaccgagactgtttaa

SEQ ID NO: 5 - DNA, 1665 bp, synthetic construct
  source 1..1665: mol_type = other DNA
atgtacaaggtccataacccctacctcacggcggcggtagccaccatgggcggaatgctctttggtttcgatatctcgtccgtgtcggcctttgtgggcgaagataactacatgaactactttggtcatcccacctccttccagcagggaggtatcaccgcctccatggccggaggatccatgctgtcgtgtgcgtttgccggctacatttccgaccgggttggccgaaagcccaccattcaatttgccgccgcctggtggatggttggcgcctccattcaatgctctgcccagaatatgggccaactgattgccggccgggccatttccgggcttggaatcggcctcggctcgtcccagatccccgtcttcatctccgagttgtcccccaagaagatccgaggccggctcgtcggctgcttccaatggtccgttacctggggtattctcatcatgttctacatttcgttcggctgctcatacatcaagggccactcgtccttccgactggcgtggggcatccagctgattccaggagccatgttggcgttcggaatgatgctgctggacgaatcgccgcgatggctggcgtccaaagaccgctgggaagaggccatccagatcatccgctccatcaatgccaactacggatccgaggaggacattctcatggaaatcgaagatctgcgagaggtggtgcgaatcgaccacgagtccaagtcggtcaccatctgggacctgttccgaaaagactccatcaaccgaaccatggtcggagtgtgggcccagatctggcaacaactgactggaatgaacatcatgatgtactacgtggtcatcatcttcaaaatggccggctactcgggcaaaagtgccgtcattgtctccggctccatccagtacatcatcaacgtggtcatgaccatcccggcgctgcttttcattgataaaatcggacgaagacccctgctcctctgtggaagcatgctcatggccacctggctgctagctgtcggaggaatgctaggagcctacggaatccaaatgccccaaggtctaccggcagtaccctccaaaaaccaggcagcggacccctacaccaccatctacattcccgacaaccaggcgccggcccgaaaggccattatcgcctgttgctatctgtttgtcgcctcttttgcacccacctggggccccggcatctggctctactgctccgagatcttccccaacaaacaacgagcgctggccaactcgctgaccgccggcgccaactggggcttcaactttgccctggccctgtttgtgcccaccgccttcaagaacatcaactggaaggtgtacatcatcttcggtgtcttctgcatcgtcatgtccatccacgtcttcctgctcttccccgaaaccaagggcaagagtctggaggtgattgaccagatgtgggacgcccgcgtgcccgcttggaaaaccgcctcctgggtccccgaccacatgccttctcattacgcaggggaccaggaggaaaagcccaccgacgaactggccgaggcgccgtttcacgaggagaatgccccggtgaacaccgagacccctcctcatgaggatgagcccacttttgcggagaccgagcccaagacccagtatcctggaactgagcatgtctaa

SEQ ID NO: 6 - DNA, 930 bp, synthetic construct
  source 1..930: mol_type = other DNA
atgacttctctcgactttactatgaacaacggcaagaccattcctgccatcggtctgggaacctggaagtccacaaccgaggaggttgctggagccgtggagtgcgctctcaccgagggcggctaccgacacattgacaccgcctttaactaccgaaacgaggacgccgtcggccttggcatcaagcgagccatggagaagggtgtcaagcgagaggatatctttgtcaccaccaagatctgggtcacttaccacgaccgagtcgaggagaacttggatctgtcgctcgagcgacttggcctcgactacgtcgacatgctgctcatccactggcccgttcctctcaaccctaacggtaacgaccccgtctaccccctgcgagaggacggatctcgagatatcgatgaatctggatctcagcccaagacctggaagcagatggaggctgttctgaagaccggtaagaccaagtccatcggtgtctccaacttctctatcccctacctcgaggagctcctgaaggaggccgaggttgtccctgctgtcaaccaggtcgagctgcaccctctgctgccccagctcgagcttatggagttctgcaagaagcacaacatcgtcatgaccgcattttcgccctttggttctgttggaggtcccctgctcaagaacgagctggttctcaagcttgccgacaagtacaagaccacccccggcggtattctgacctcctaccacattggaaacggcactgttgtgatccccaagtccgtcaccaactctcgaatcatcgagaacggaaagtccgccgtcaccctcaatcaggaggacctcaagtctctcaatgacctgcacaagaccgagggtatccaccgaacctccaagcccaagtggggtgtcgatctcggattccccgactttgacttctgctaa

SEQ ID NO: 7 - DNA, 930 bp, synthetic construct
  source 1..930: mol_type = other DNA
atgacttccatcgactttaccatgaacaacggcaagaccatccctgccatcggtctgggaacttggaagtccaccaccgaggaggttgccggcgccgtcgagtgtgctctcaccgagggaggttaccgacacattgacaccgcattcaactaccgaaacgaggacgctgttggtctcggtatcaagcgagccatggagaagggcgtcaagcgagaggacatctttgtcaccaccaagatctgggtcacctatcacgaccgagtcgaggagaacctgaacatgtctctggagcgactgggtcttgactacgtcgacatgctgctgatccactggcccgttcccctcaaccccaacggtaacgaccccgtctaccctctgcgacccgacggatctcgagacattgacgagtccggctcccagcccaagacctggaagcagatggaggctgttctgaagactggcaagaccaagtctatcggtgtctccaacttctccatcccctacctgcaggagctgctcaaggaggccactgttgtgcccgccgtcaaccaggtcgagctgcaccctctgctgccccagctggagctcatggagttctgcaagaagaacaacatcatcatgaccgccttctctcctttcggatctgtcggaggccctctgctcaagaacgagctggttctcaagctcgccgacaagtacgagacctctcccggcggcattctcacctcctaccacattggcaacggcactgttgtcattcccaagtccgtcaccaactctcgaatcatcgagaacggtaagtctgccgtcaacctgtccaaggaggacctgcaggctctcaacgatctgcacaagaccgagggtatccaccgaacctccaagcccaagtggggagttgatctcggtttccccgactttgacttttgctaa

SEQ ID NO: 8 - DNA, 930 bp, synthetic construct
  source 1..930: mol_type = other DNA
atgacttctatcgacttcactatgaacaacggcaagactatccctgccatcggtctgggaacgtggaagtcgaccaccgaggaggttgccggagctgtcgagtgcgctctcaccgagggaggttaccgacatattgacaccgccttcaactaccgaaacgaggacgcggtcggtctcggtatcaagcgggccatggaaaagggtgtcaagcgagaggacatctttgtcaccaccaagatttgggtcacttaccatgaccgagtcgaggagaacctggatctgtcgctggagcgactgggtctcgattacgtcgacatgctgctcatccactggccggtgcccctcaaccccaacggaaacgaccccgtctaccctctgcgaccggacggatctcgagacattgacgagtccggctcgcagcccaagacctggaagcagatggaggccgttctgaagaccggcaagaccaagtccatcggagtctccaacttctccattccctacctggaggagttgctcaaggaggccgaggtggtccccgctgtcaaccaggtcgagctgcaccctctgctgccccagctcgagctcatggagttctgcaagaagaacaacatcatcatgactgccttctctcctttcggatctgtcggaggccctctgctcaagaacgagctggttctcaagctcgccgataagtacgagacctctcccggcggtattctcacttcctaccacattggtaacggcactgttgtcattcccaagtccgtcaccaactctcgaatcatcgagaacggtaagtccgccgtcaacctgtccaaggaagacctgcaggctctcaacgatctgcacaagaccgagggtatccaccgaacttccaagcccaagtggggagttgatctcggtttccccgactttgatttctgttag

SEQ ID NO: 9 - DNA, 930 bp, synthetic construct
  source 1..930: mol_type = other DNA
atgacttctatcgactttaccatgaacaacggcaagaccatccctgccatcggtctgggaacctggaagtccaccaccgaggaggttgccggtgctgtcgagtgcgctctcaccgagggtggctaccgacacattgacaccgccttcaactaccgaaacgaggacgccgtcggccttggtatcaagcgagccatggagaagggtgtgaagcgagaggacatctttgtcaccaccaagatctgggtcacttaccatgaccgagtcgaggagaacctcaacatgtctctcgagcgacttggtctcgactatgtcgacatgctcctgattcactggcctgttcccctcaaccccaacggtaacgaccccgtttaccctcttcgagaggacggatctcgagacattgacgagtccggctcccagcccaagacctggaagcaaatggaggctgtcctcaagactggcaagaccaagtctatcggtgtctccaacttctccatcccctacctcgaggagctgctcaaggaggccgaggttgtccctgctgtcaaccaggtcgagctgcaccctctgctcccccagctcgagctcatggagttctgcaagaagaacaacatcatcatgactgccttctctccctttggttccgtcggaggccctcttctcaagaacgagctggttctcaagcttgccgacaagtacaagacctcccccggtggcattctcacctcttaccacattggtaacggcactgttgtcatccccaaatccgtcactaactctcgaatcatcgagaacggtaagtctgctgtcaccctgtctcaggaggacctcaaggctctgaacgacctccacaagaccgagggtatccaccgaacctccaagcctaagtggggtgttgacctcggattccccgacttcgacttctgctaa

SEQ ID NO: 10 - DNA, 732 bp, synthetic construct
  source 1..732: mol_type = other DNA
atgtcctccgaactgcctcctcttgagcaggccaagcgaatcgccgcccaccaggccgtggagcaacactaccccaaggacgccaaggtcgtgggcattggctcaggatccaccgtggtctacgttgccgagaagattgcgtcgttgcccaaggagctcaccaaggacaccgtgttcatttctacgggtttccagagcaagcagctgatccagaacgccgggttgcgactgggatgcatcgaccagtactccaacggagatctggacgtggcgtttgacggcgccgacgagaccgaccctcagctcaactgcatcaagggcggaggagcatgcctcttccaggaaaagattgtcgccgagtgtgcccgcaagtttgtcgtggtggccgactaccgaaaacagtccaaggctctgggcaccgtgtggatccagggtatccccattgaggtggtgcccgacgcctacaacaaggtaattgctgatctcaagaagatgggcgcccagtccgcggtgctgcgacccggctctcccggaaaggcgggccccatcatcaccgacaatggcaacttcattgtcgacgcctactttggcgagatccaacccgacgccgtcaaggacctgcacatcaagatcaagctgctgttgggcgtcgttgagaccggcctcttcactaacgcggacgtagcgtactttggaaacgccgacggaaccatctccaccattaccaagtaa

SEQ ID NO: 11 - DNA, 750 bp, synthetic construct
  source 1..750: mol_type = other DNA
atggtccagccaatcatcgccccctctattctttcctcagactttgccaagctcggcgcacaatcgcgaatgatggtcgactacggagcggactggctccacgttgacatcatggatggccactttgtgcccaacatttcgtttggccccaccgtggtcaagtgcctgcgagccgaacttctcaagggcgaggcgttttttgactgccacatgatggtcactgatcccctacgatgggtcgagcccatggccgacgccggagccgaccagtacaccttccactacgaggcatgctacgaccacgaggaggtcatcaaggccatcaaggccaagggaatgaaggccggcattgctatcaagcccgccacctcggtggaggttgtctaccctctggttgaccagcttgatctggttctcgtcatgactgtggagcctggctttggaggccagaagttccagcaggacatgatgcccaaggtggctgctctgagagagcgataccccaacctcaacatcgaggtcgatggtggtcttggcccccagaccgtcgatgacgccggagctgctggtgctaatgtcattgtcgccggtacctctgtctaccactccgactctcctccccgaaccatcaaggagatgcgagacaagctcatcacctatcagaaggacattgagatggccgccgagcaccccgagatcgttaccatccagcaagagattctgcctgtccccgctgctgcctctgcctaa

SEQ ID NO: 12 - DNA, 1440 bp, synthetic construct
  source 1..1440: mol_type = other DNA
atgacaatcactctgagtcagaaggttggcgagttcgcctccgagttcgccatcgatggcagctatgttcagcaaggcgtcaaacacttcgtcgatcagacagcacaggccctctcagcaaacatggaaagagcaatgcccatgattccctcgtttgtcacccgaattcccacagggaaggaaaagggtgtccagttggcagttgacctgggaggcactaacttccgggtctcatccgtgcacctgaatggtgaccacacccacaaaacggtggcatcaaaagttgctgtcagcgatgacctcatgactggcactcaagctgacttctttggatttttggcccagcaagtgaaagatttcttaacagaacaccactcagaacatttcagccagactgagcctttgcgaatgggagtgaccttctcgttccccgtcaatcaggtagctttagacaagggctatcttttgagatggaccaagggttatgatatcaaggatgctgttggaaaagacgtcgtggagctatttcagatccagctcgatgctcttgacctgaatgtgaagattgtggctctggtcaacgacaccgtgggcacactattggcgcgagcttacgcctctaaatccgacgatggtaagcataagcagaccttgatcggcgcaatctttggcaccggaaccaatggtgcttactcagaaagacttaagcgggtccccaagtttaccgctgcgaattaccctgaagttttcaagcgctactcaccagagcacatcgcgaaccaaactatgattatcaataccgagtggggatcttacgataatgacattgacgtcctcccaaacaccaaatgggatattgacttggacgaaaacactcacaacaagtcttttcacatgtttgaaaagcggatttctggaatgttcctgggtgagctgttgcggctctgtcttttggatttgaagagtgagggcctacttcttcaatccacaaagttggaaggaaaccctgtttggaagggatttggaatggacacttcggtcttgtcattcattgaagcagccactgaggcacagatcgagacaggaaagggcgactatgagaaaatcatcaaggccatgggtctggccgccaaggacatcactcctgaacaagttgctgccctgaaacccctggttacggctattggtagacgatctgcgtatctatctgccattcctttagctgccattactcttcatactcacgctttggagaaattcgacgttgttgacattggagttgacggttctgtcttccagttttaccccaaatttgctgatatggtgaaagaagcgatgggctatgtcgagggtatttctgttgaccgtatcgacatcggccacgcagaagatggctcaggggtgggagctgccttggctgcttacccggacgactcatag

SEQ ID NO: 13 - DNA, 1566 bp, synthetic construct
  source 1..1566: mol_type = other DNA
atgaattacgcgcgcgacaacgactactgcgagtccgccgacgggcaaatcggctcgtcggactcaaatgaggtgtctgagatgggtcacaagaaggaacttgaacaggtcttttctaccgccacagcattcgatctcgagtcagaagacacgcagaaagagctggaccgcatctatcgcaaactggaccgtcgaatcatcccgcctctgtggatcctgtactttctgacgtcattcggaagcagtgcttacggagtggcgctgaccatgaacatggacaagggccattcgctgtctcagaccctgaatctgtcggcccaccagctctcggttgcctccgctctctattatgtcggttacattgtctttgatgtgcccatcaatctggtgatgaccaaagtggccccccatgcgtggttatctcgtatcgtcatttccgtgggcgttgtttacgcatgttatgcggctctgaacaatgccggtggcgtcattgccatccgtctcatttcgggcatctgtggagccggcacttggcccggactggcctactatgtgtcgttgtggtatcctgctcaccggaccgcgcgacgtatcggctactatttcaccgctgcccaggtgtctgccgccaccgcgggtctgttttccgccgcgttccagaccattgacggcaaccgtggcctcaagggcttccaatggttctttctcgtctacggagtgttcaccgtcactctaggagtctccctactgtggtggttgccaggccgaccgtcggagttcaacgatctctacgacgagtcccataagattgaccagttggccaagacgtcggttctcaagtccctgctcaacggcaccttctccaaacacccgctttctccgctcgaacaacagttgcacgccgccgacatgtctcaacggtacctcaacgtggcctggactctcaaggacctgctgagagtgtttatggacatccgaatctggccgctggtcattatgtactttggagtggtcggaaccggctacggtattgtggtggccggtacctctatcatcaagtccaccaacgagtctctgacccccattcaactgtctctgcttttctgtcccatctgggtgtgcgatcttctcgccattcttgcaatcactccgctctctgaccgctacaagaaccggccgctgtttttctgcgcgtctacagtggtcattattgttggaatggtcgtcaacacctatgcgcctgggtcgtggccccgatatgtgggactacttatcaccggactcggtctgggccccacagtgcccatctgcatgacctgggcggctgagatcttcatgcccaagtaccaggacgtcggggtcgccgccacttcggctctagtttctggcctgggaaatctcggcagtgtcgtcaccacctacgcactgtacaaaggctggagtgacgatgtggaccggggcttcaagtactccaatatgaccgttgttggcattttgggtgcgtcgattattgccgctttgctcctgggctacactgagaggagacggttgcgtggccagcaatga

SEQ ID NO: 14 - DNA, 2862 bp, synthetic construct
  source 1..2862: mol_type = other DNA
atgattgaaggaatctcctttgcgtcgtttgtgacacacgaaaaacccaagtttgtgcgcgccctggacttctacaaggccctgggcttcctccccaccaaggagtacaagcacggaacagaccaccatgccaccgacgaagaaggcgcgggctcgatccaggaggtctggctcacgtcgtcgcgagccggtgtgccgtctgtgaccgtcaaattgcgactctcccgccacggaaacgaacacgtgtctctccccaacctcaaacacgactggcggtcgctggtgccctcgctggtgtactacgcccccgacctggacgcggtgcgagccgcaatcaccccgttcttacacgaggaccactccacgcttctggaacgcccttcccacacaaacttcatcgagctgtacgccattgaccccatgggaaacctcgtgggcttttcccgacgagaaaacccttactcgtcagcgatgcagaaacccttctctgctgacgacattggcccgcagaacttctcaaagcccaatgaaaccaaaatcaagggcaagaagcgtatcggtgtcatgacctccggaggagacgcccccggcatgtgtgcggccgtgcgagctgtggtccgagccggaattgcccgcggctgcgaggtctacgctgtccgagagggctatgagggtcttgtcaagggtggcgacctcattgagcccctgtcatgggaagacgtacggggctggctctccctgggaggaacccttattggaactgctcgatgcaaggagttccgagaacgagagggccgactggcaggagccctcaacatggtcaagaacggcattgacgcgctcattgtcattggcggagacggctcgttgaccggagccgatctgttccgagaagaatggccgtctctaatcgaggagctagtcaccaatggctccatcaccgccgagcaggccgaacgacaccgacacctcgacatttgcggtatggtaggctccatcgacaacgacatggctaccaccgacgtcaccattggtgcttactcatcgctcgaccgaatctgcgagctggtagacttcatcgatgccaccgcccagtcgcactcgcgagcctttgtcgtcgaggttatgggtcggcactgtggctggctggctctcatggccggcactgctaccggcgccgactacatttttatccccgaggctgctcccgatgctactcagtgggccgagaagatgacccgtgtcgtcaagagacaccgaagccagggcaagcgaaagaccgtggtcattgtcgccgagggcgcaatcgactcggacctcaaccccatcactgccaagatggtcaaggatgtgctagacggcattggactcgatactcgaatctccaccctgggtcacgtacagcgaggaggtcccccagttgccgctgatagagttctggcttcactgcagggtgtggaggctatcgacgccatcctgtctctcaccccagagacgcccagtcccatgattgctctcaacgagaacaaaatcacccgcaagccgctcgtggagtctgtagctctcaccaagaaggttgccgatgccattggcaacaaggactttgccgaggccatgcggcttcgaaaccccgagtttgtggagcaattgcagggtttcctgctcacaaactctgctgacaaggaccggccccaggagcctgccaaggatcccctgcgagtcgccattgtgtgcactggcgctcctgctggcggaatgaacgctgccatccgatctgctgttctgtacggtcttgctcgaggccaccaaatgtttgccatccacaatggatggtccggcctcgtcaagaatggtgacgacgcggtgcgggagctgacttggctcgaggtcgagcccctgtgtcagaagggtggctgtgagattggtactaaccgatctctgcccgaatgtgatcttggaatgattgcataccactttcagcgacaacggtttgacggtctaatcgtcattggtggttttgaggctttccgagcgctgaatcagctcgacgatgcccgtcacgcctaccctgctcttcgaatccccatggttggtattcctgccaccatttcgaacaacgtgcctggaacggactattctcttggagccgacacttgtctcaactctctggttcagtactgcgacgtgctcaagacgtctgcttctgccactcgtctgcgtctgtttgtggtcgaggtgcagggtggaaactctggttacatcgccaccgtggctggtttgatcaccggcgcctatgtggtgtacacacccgagagcggtatcaacctgcgacttcttcagcacgacatttcctaccttaaggatacttttgctcatcaggcggacgtgaaccgaaccggaaagctgcttctgcgaaacgagcggtcatccaacgtgttcaccactgatgtcatcaccggcatcatcaacgaggaggccaagggttcatttgacgcgcgaaccgccatccctggccatgtgcagcagggaggacacccctctcctaccgatcgagtgcgtgctcagcgatttgccatcaaggccgtgcagtttattgaagagcaccacggctccaaaaacaatgccgatcactgtgtgattctcggtgtgcggggctccaagttcaagtacacctctgtgtcgcatctgtacgcccataagactgagcacggggctcgacggcccaagcattcctactggcacgcgattggcgacattgccaacatgctggtgggtcgaaaggcgcctcctctgcccgagactctcaacgacgagattgagaagaacattgcgaaggagcagggtattattgatccttgttag

SEQ ID NO: 15 - DNA, 1197 bp, synthetic construct
  source 1..1197: mol_type = other DNA
atgcctgttactgacgtccttaagcgaaagtccggtgtcatcgtcggcgacgatgtccgagccgtgagtatccacgacaagatcagtgtcgagacgacgcgttttgtgtaatgacacaatccgaaagtcgctagcaacacacactctctacacaaactaacccagctcttcgagtacgcccgagagcacaagttcgccctccccgccgtcaacgtgacctcttcgtccaccgttgtcgccgttcttgagtctgcccgagccaacaagtcccccgtcatcatccagatgtcccagggtggcgctgcctactttgctggcaagggtgtcgacaacaaggatcagaccgcctccatccagggagccattgccgctgcccagttcatccgaaccattgctcccgtttacggcattcccgtcatcgtccacaccgaccactgtgcccgaaagctgctcccctggctcgacggtatgctcgacgccgatgaggagtacttcaagactcacggtgagcccctcttctcttcacacatggtggatctctccgaggaggagcaccccgagaacatcgccaccaccgccgagtacttcaagcgagccgccaagatgaaccagtggctcgagatggagattggtgtcaccggaggtgaggaggatggtgttgacaacactggtgtcgacaactccaagctctacacccagcccgaggacatttacgccgtctactccgccctcgctcccatctcccccaacttctccattgccgctgccttcggtaacgtgcacggtgtctacaaggtcggcaacgtcaagctgcaccccgagctcctcgagaagcaccaggcttactccgccgagaaggttggctctcccgagaagcacggcaacggaaagcctctcttcctcgtcttccacggtggatccggttcttccgactccgactacaagattgccattgataacggtgttgtcaaggtcaacctcgatactgatctgcagtacgcttaccttgttggtatcagagactacattctctccaagaaggactacctcatgcagcaggtcggcaaccccgagggtgatgacaagcccaacaagaagtacttcgacccccgtgtctgggtccgagagggtgagaagaccatggccaagcgactcgactctgcctttgaggttttcaacgccaagaacaccttgtaa

SEQ ID NO: 16 - DNA, 1359 bp, synthetic construct
  source 1..1359: mol_type = other DNA
atgtctcccaaaacacccgtagctcacccccaccgcccatactcatcatacaactccctcaaacatcatcaagccaacaacccgcctccgcattaccctcagcactatccaagtcccccaagcctaaccacgagcccccatgacaagatggaagaggggcagagagacatatggtactatgaagatgactcccagcagcaacagcaacagcagccttcccagatccagcatgcccagcagcagcagcagcctcctcaaactccttcaaacccccataaccctctgatgcccccgtctcagccccaactgtcccagggacaccctcatcggcaatcccagcccctggtgccgccttccagacactcgtcttccagttcatccgaagcccaggccggatcagctgggggcgtggctcactcggcagccgcagcggccgcagcagcccagtacgctccattcagagctcgaataaccactactttgtgggaagatgaagggacgctgtgcttccaggttgaagcacgaggcgtgtgcgtggctcgtcgagaagataacaacatgatcaatggcacaaagctgctcaatgtggtgggaatgactcgaggccggcgggatggcattctcaagacggagaaaatcagacacgtggtcaaaattggcgccatgcatctcaagggcgtgtggattccgtatgagcgagcgctggcgtttgctcaacgggaacggatcgttgacgtgttgtaccctctgtttgtgtccgacattaaagaactgctctaccaccctgtcaactactcgcggacagccctggttctggcggtcgccgagcggcgtaaaatcgaacaacaacgacttcaacaattgcaggagatccacgccggcatggacacccagctattgacccagggagttgctcagccgggagtccagcaacagggtgctcaggtcgctggccaacctcagatcaccccttccgcttccactccttactctcagtatcaaacccctccccagtcccagatgaccatgggacaataccagtcttcgcagcactcgcagcagcagcaacagcctgggacacttggccagcccatcaccaccggagacccctaccccctcccgtcgcttccctcgacacatgaaaacaacatgcgccatgcccaaatttactatcatcaacaatacggctacccttccgaccagtctctgccaccgcccttgcgccttcaccagccccagcccggccagactccccaacaacagccgcagcagccgcagcatcaacagcaaccgcagcatcaacagcagcaattgcctcctcctccgcattacgggacccccggaaactacggtagctaa

SEQ ID NO: 17 - DNA, 1350 bp, synthetic construct
  source 1..1350: mol_type = other DNA
atgtacccctacagtcagcagccccagtactcgtcgtactacggcaacgcgtcgcaccagtaccctgggtaccagcaggccaacccctccggctctccccaggccactgcttccccccagcagcatgctcccggtggccagcccggtggtgccaacacccacctcatgtaccagcagcccatgtatggctactcgcagccacacgtgcctgcctcgtcccagagtggcggctccgccccttacggcatctcctcgcagcttgcaggcgccgtgcttgccgctgcagccctccccggcggacccttgcccatcaccattacagaccccaccggtcagaaccccccaccgggcgtcaagcccaaggtgaccaccacgtcatgggaggacgagggaacactgtgtttccaggtcgaggcccgaggcatctgcgtggcgcgtcgagaggacaacgacatgatcaacggtaccaagctcctcaacgtggccggcatgacccgaggccgacgagacggtattctcaagggagagaagctgcgtcatgtggtcaaggccggcgccatgcatctcaagggcgtgtggatcccttatgatcgagccctcgagtttgccaacaaggagaagatcatcgatctgctcttccccctgtttgtgcgagacatcaagtccgtgctgtaccaccctgccaactacgcccgaaccgtgcagcccatgacttctgtggatgtcaagcgagaggaggacggtgttgccggccagcagcagcagcagcagggtcagcagcagcagggtcaggtccagcctggccagcagcccggccagcagcagcagcagcctggtcagcagcccggtgtgcagcagcagcaacaaggtggcgcccagcagcagcctccggcctcgtaccgatcgcaccacctggaccatctgcagtccgtcgagcggtccacctccacgccgcctcctgcttcccagtcgtcctactactaccagcactcgagcacctcggacggctttgagagccccggtccctcgtccttttcgacccccggctcgcgcatcaacaccccctccaccgccgagtactaccagcagggcaaccccgcctacaaggctgctcctacttctgcctctgtgagcaaccccccggcctcctcttctgcgcagtctactccccaggcccagcccgcccagcctcctcaggctaacggcgctgtcaacggccaggccaacggcaagggctactccaactacatgccccagatctaccagagccagactgcctcgcccgccgcctccgagccccaggccaaggaggaggaaaaggtgtctctcccttctatgcaccatgagtag

SEQ ID NO: 18 - DNA, 6227 bp, synthetic construct
  source 1..6227: mol_type = other DNA
cagatcttggtggtagtagcaaatattcaaatgagaactttgaagactgaagtggggaaaggttccgtgtgaacagcagttggacacgggtaagtcgatcctaaggggtggcataactgtcgcgtacggcccgataagggccttctccaaaagggaagccggttgaaattccggcacttggatgtggattctccacggcaacgtaactgaatgtggggacggtggcacaagtcttggaaggagttatcttttctttttaacggagtcaacaccctggaattagtttgtctagagatagggtatcgttccggaagaggggggcagctttgtcccctccgatgcacttgtgacgccccttgaaaacccgcaggaaggaatagttttcacgccaagtcgtactgataaccgcagcaggtctccaaggtgaacagcctctagttgatagaataatgtagataagggaagtcggcaaaatagatccgtaacttcgggataaggattggctctgggggttggtggatggaagcgtgggagaccccaagggactggcagctgggcaactggcagccggacccgcaagcttctgaggtgtctcacaagtgccgtgcagtcccgcccccacttgcttctctttgtgtgtagtgtacgtacattatcgagaccgttgttcccgcccacctcgatccggctgaggtgtctcacaagtgccgtgcagtcccgcccccacttgcttctctttgtgtgtagtgtacgtacattatcgagaccgttgttcccgcccacctcgatccggctgaggtgtctcacaagtgccgtgcagtcccgcccccacttgcttctctttgtgtgtagtgtacgtacattatcgagaccgttgttcccgcccacctcgatccggctgaggtgtctcacaagtgccgtgcagtcccgcccccacttgcttctctttgtgtgtagtgtacgtacattatcgagaccgttgttcccgcccacctcgatccggcacgggcaaaagtgcgtatatatacaagagcgtttgccagccacagattttcactccacacaccacatcacacatacaaccacacacatccacaatggaacccgaaactaaggagctcatgtccactagtgctatgaccgacgattatcccatggctgaggtctccattactgaggagaccaagatgcctgagcctgccaagaaatcgcagcttcttgtcattctgctctgtctctttgccgccatgggcggttttgtctttggctatgacactggtaccatttctggcttcatcaacatggaccctttcctagagagctttggagaagccgatgagtctggcgttttctttctatcccgaatccgagctgggctcattgttggactcttttccattggtgctttccttggcacccttcttggtggcttccttgctgacaaggttggtcgaaagaagggaattgtctatgtcgccatggtctacattgttggtatgcttatccagatcacagccttcacagcctggtaccagattgccattggtcgtgtcatcggtggtgttggtattggtgcactctctgtgctggtccccatgttccagtcagagactgctccccagaatcttcgaggcgctcttgtctcttctttccagctgtttattactctcggcatcttcattggatttagtgtttgctacgctaccaagtcgcggctcgataccggtgcctaccgaatccccatgggtctttgcttcgcctgggcttttatccttctcattggcatgtgcttcatgccagagtcccctcgattcctggtttcaattggccgcattgaggaggcccgaaaggctatggccatgacaaaccaggttcctctccacgacgctgtcattgatgaggaactcaaggctattgagaactctgtcattagggaaaagagtgctggtaaggccacatggaaagaacttttcactggcgagccccgaatgggctaccggcttacccttggtattctcgtccaggttctccagcagctctgtggtgccaactactttttctattacggaacttctatcttcaaggccattggaatgtcggattctttcgctacttcgatgatctttggcggaatcaacctcctgtctacctttggaggtctctacattgttgatcgattcggtcgacgaaagtgccttcttggtggagccatggtcatgtttgtctgtttcctcgtctattctactgtcgggttcgctgctctttatcccaacggtgataccacccttcccgccagaaaggctgttggtgatgtcatgattctgttcacctgtatcttcatcgccgctttcgcctctacgtgggcccctattgccttcgtcgttgtttctgagaccttccctctcagaatgcgatcaaagggaatggctgttgctactggaggaaactggatgatcaacttcctcgtttctttcctgactcccttcatcaccagttccattgggttcaagtacggttatgtcttcactgcttgcattggtttcgccattatctttgtcttcttcttcatccctgagaccaagggtctgtcccttgaggatgttgacgagctgtacgcttcaggtgtcagtgctcgaaactcccccaactgggttccttcttccaagaagcaggaagtcaacgaacctaagaacagtatgaccgacttctccgagtcctcttagctcgaggcaattaacagatagtttgccggtgataattctcttaacctcccacactcctttgacataacgatttatgtaacgaaactgaaatttgaccagatattgttgtaaatagaaaatctggcttgtaggtggcaaaatcccgtctttgttcgtcggttccctctgtgactgctcgtcgtccctttgtgttcgactgtcgtgttttgttttccgtgcgtgcgcaagtgagatgcccgtgttcgaatacggtagtcgcacggaatcgatcctacgtatgggccctgcatctgatctgtcatataacttcgtataatgtatgctatacgaagttataaggagtttggcgcccgttttttcgagccccacacgtttcggtgagtatgagcggcggcagattcgagcgtttccggtttccgcggctggacgagagcccatgatgggggctcccaccaccagcaatcagggccctgattacacacccacctgtaatgtcatgctgttcatcgtggttaatgctgctgtgtgctgtgtgtgtgtgttgtttggcgctcattgttgcgttatgcagcgtacaccacaatattggaagcttattagcctttctattttttcgtttgcaaggcttaacaacattgctgtggagagggatggggatatggaggccgctggagggagtcggagaggcgttttggagcggcttggcctggcgcccagctcgcgaaacgcacctaggaccctttggcacgccgaaatgtgccacttttcagtctagtaacgccttacctacgtcattccatgcatgcatgtttgcgccttttttcccttgcccttgatcgccacacagtacagtgcactgtacagtggaggttttgggggggtcttagatgggagctaaaagcggcctagcggtacactagtgggattgtatggagtggcatggagcctaggtggagcctgacaggacgcacgaccggctagcccgtgacagacgatgggtggctcctgttgtccaccgcgtacaaatgtttgggccaaagtcttgtcagccttgcttgcgaacctaattcccaattttgtcacttcgcacccccattgatcgagccctaacccctgcccatcaggcaatccaattaagctcgcattgtctgccttgtttagtttggctcctgcccgtttcggcgtccacttgcacaaacacaaacaagcattatatataaggctcgtctctccctcccaaccacactcacttttttgcccgtcttcccttgctaacacaaaagtcaagaacacaaacaaccaccccaacccccttacacacaagacatatctacagcaatgctccgaatcgctaaagaagctctgacctttgacgacgttctcctcgttcctgctcactctaccgttctgcccaatactgctgacctctccacccagctgaccaaaactattcgactgaatatccctatgctctccgctgctatggataccgttaccgaagcccgactggctattgctctggctcaggaaggcggtatcggctttatccacaaaaacatgtccattgaacgacaggctgaagaagttcgacgagtgaaaaaacacgaatctggtgtggtgactgatccccagactgttctgcctaccaccaccctgcgagaagtgaaagaactgaccgagcgaaacggttttgccggctatcccgtcgttaccgaagaaaacgaactggtgggtattatcaccggtcgagacgtgcgatttgttaccgacctgaaccagcccgtttccgtttacatgacccccaaagagcgactggtcaccgtgcgagaaggtgaagcccgagaagtggtgctggctaaaatgcacgaaaaacgagttgaaaaagccctggtggttgatgacgagttccacctgatcggcatgatcaccgtgaaagacttccagaaagccgaacgaaaacccaacgcctgtaaagacgagcaaggccgactgcgagttggtgctgccgttggcgctggtgccggtaacgaagagcgagttgacgccctggttgccgctggcgttgacgttctgctgatcgactcctcccacggtcactctgaaggtgttctgcaacgaatccgagaaacccgagctaaatatcccgatctgcaaattatcggcggcaacgtggctactgctgctggtgcccgagccctggctgaagctggttgctccgccgttaaagtcggcattggccctggctctatctgtactactcgaatcgtgactggcgtcggtgttccccagattaccgctgttgctgacgctgttgaagccctggaaggcaccggtattcccgttatcgctgatggcggtattcgattctccggcgacatcgccaaagctatcgccgctggcgcttccgccgtgatggttggttccatgctggccggtactgaagaatctcccggtgaaatcgaactctaccagggccgatcttacaaatcttaccgaggtatgggttccctgggtgccatgtccaaaggttcctctgaccgatatttccagtccgataacgctgccgacaaactggtgcccgaaggtatcgaaggtcgagttgcctataaaggacgactgaaagagatcattcaccagcagatgggcggcctgcgatcctgtatgggtctgaccggctgtggtactatcgacgaactgcgaactaaagccgagtttgttcgaatctccggtgccggcattcaggaatcccacgttcacgacgtgaccattactaaagagtcccccaactaccgactgggctcctgatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgataacttcgtataatgtatgctatacgaagttatcagcatcgtaatagcctccaagagggcgccggtgaaataccactacccttatcgtttctttacttatttagtaagtggaagtggtttaacaaccattttctagcattcctttccaggctgaagacattgtcaggtggggagtttggctggggcggcacatctgttaaaagataacgcagatgtcctaagggggactcaatgagaacagaaatctcatgtagaacaaaagggtaaaagtccccttgattttgattttcagtgtgaatacaaaccatgaaagtgtggcctatcgatcctttagttgttcggagtttgaacctagaggtgccagaaaagttaccacagggataactggcttgtggcagtcaagcgttcatagcgacattgctttttgatccttcgatgtcggctcttcctatcataccgaagcag

SEQ ID NO: 19 - DNA, 18 bp, synthetic construct
  source 1..18: mol_type = other DNA
accgtggagaaaagcccg

SEQ ID NO: 20 - DNA, 20 bp, synthetic construct
  source 1..20: mol_type = other DNA
aagaggatcagacccagtcg

SEQ ID NO: 21 - DNA, 19 bp, synthetic construct
  source 1..19: mol_type = other DNA
gacgattatcccatggctg

SEQ ID NO: 22 - DNA, 23 bp, synthetic construct
  source 1..23: mol_type = other DNA
tcgaccaaccttgtcagcaagga

SEQ ID NO: 23 - DNA, 21 bp, synthetic construct
  source 1..21: mol_type = other DNA
gcgcttagttgcacgtcatag

SEQ ID NO: 24 - DNA, 21 bp, synthetic construct
  source 1..21: mol_type = other DNA
gtgccttgtgtcagggtcttg

SEQ ID NO: 25 - DNA, 21 bp, synthetic construct
  source 1..21: mol_type = other DNA
tccatggccggaggatccatg

SEQ ID NO: 26 - DNA, 22 bp, synthetic construct
  source 1..22: mol_type = other DNA
catggctcctggaatcagctgg

SEQ ID NO: 27 - DNA, 19 bp, synthetic construct
  source 1..19: mol_type = other DNA
ggttgctggagccgtggag

SEQ ID NO: 28 - DNA, 19 bp, synthetic construct
  source 1..19: mol_type = other DNA
gctgagatccagattcatc

SEQ ID NO: 29 - DNA, 20 bp, synthetic construct
  source 1..20: mol_type = other DNA
caacggcaagaccatccctg

SEQ ID NO: 30 - DNA, 19 bp, synthetic construct
  source 1..19: mol_type = other DNA
cagtcgctccagagacatg

SEQ ID NO: 31 - DNA, 22 bp, synthetic construct
  source 1..22: mol_type = other DNA
ggaggttgccggagctgtcgag

SEQ ID NO: 32 - DNA, 24 bp, synthetic construct
  source 1..24: mol_type = other DNA
actcgtcaatgtctcgagatccgt

SEQ ID NO: 33 - DNA, 22 bp, synthetic construct
  source 1..22: mol_type = other DNA
catgaacaacggcaagaccatc

SEQ ID NO: 34 - DNA, 22 bp, synthetic construct
  source 1..22: mol_type = other DNA
tcgagagacatgttgaggttct

SEQ ID NO: 35 - DNA, 18 bp, synthetic construct
  source 1..18: mol_type = other DNA
gagcaggccaagcgaatc

SEQ ID NO: 36 - DNA, 20 bp, synthetic construct
  source 1..20: mol_type = other DNA
gtcggtctcgtcggcgccgt

SEQ ID NO: 37 - DNA, 19 bp, synthetic construct
  source 1..19: mol_type = other DNA
ctggctccacgttgacatc

SEQ ID NO: 38 - DNA, 21 bp, synthetic construct
  source 1..21: mol_type = other DNA
ggtcgtagcatgcctcgtagt

SEQ ID NO: 39 - DNA, 23 bp, synthetic construct
  source 1..23: mol_type = other DNA
cagacagcacaggccctctcagc

SEQ ID NO: 40 - DNA, 24 bp, synthetic construct
  source 1..24: mol_type = other DNA
cgagctggatctgaaatagctcca

SEQ ID NO: 41 - DNA, 22 bp, synthetic construct
  source 1..22: mol_type = other DNA
gagatgggtcacaagaaggaac

SEQ ID NO: 42 - DNA, 22 bp, synthetic construct
  source 1..22: mol_type = other DNA
cactttggtcatcaccagattg

SEQ ID NO: 43 - DNA, 22 bp, synthetic construct
  source 1..22: mol_type = other DNA
gtctggctcacgtcgtcgcgag

SEQ ID NO: 44 - DNA, 23 bp, synthetic construct
  source 1..23: mol_type = other DNA
ttttggtttcattgggctttgag

SEQ ID NO: 45 - DNA, 22 bp, synthetic construct
  source 1..22: mol_type = other DNA
ctcttcgagtacgcccgagagc

SEQ ID NO: 46 - DNA, 22 bp, synthetic construct
  source 1..22: mol_type = other DNA
gccactggttcatcttggcggc

SEQ ID NO: 47 - DNA, 24 bp, synthetic construct
  source 1..24: mol_type = other DNA
cctcatcggcaatcccagcccctg

SEQ ID NO: 48 - DNA, 22 bp, synthetic construct
  source 1..22: mol_type = other DNA
accacgtgtctgattttctccg

SEQ ID NO: 49 - DNA, 23 bp, synthetic construct
  source 1..23: mol_type = other DNA
gcccggtggtgccaacacccacc

SEQ ID NO: 50 - DNA, 22 bp, synthetic construct
  source 1..22: mol_type = other DNA
accacgtgtctgattttctccg